(19) United States
(12) Patent Application Publication
(10) Pub. No.: US 2025/0027945 A1
(43) Pub. Date: Jan. 23, 2025

(54) ANTI-RELATED-TO-RECEPTOR TYROSINE KINASE (RYK) ANTIBODIES AND USES THEREOF

(71) Applicant: The Regents of the University of California, Oakland, CA (US)

(72) Inventors: Thomas J. KIPPS, San Diego, CA (US); George F. WIDHOPF, II, San Diego, CA (US)

(21) Appl. No.: 18/684,453

(22) PCT Filed: Aug. 18, 2022
(86) PCT No.: PCT/US2022/075147

§ 371 Date: Feb. 16, 2024

Related U.S. Application Data
(60) Provisional application No. 63/234,527, filed on Aug. 18, 2021.

(51) Int. Cl. G01N 33/574; C07K 16/40; G01N 33/573

(52) U.S. Cl. CPC G01N 33/57492; C07K 16/40; G01N 33/573; C07K 2317/24; C07K 2317/33; G01N 2333/912

(57) ABSTRACT

Provided herein are, inter alia, antibodies, which bind Related-to-Receptor Tyrosine Kinase (Ryk) with high efficiency and specificity. The antibodies and antibody compositions provided herein include, for example, novel light and heavy chain domain CDRs and framework regions and are, inter alia, useful for diagnosing and treating cancer and other RYK-related diseases. In embodiments, the anti-RYK antibodies provided herein are capable of binding a human RYK protein, but not a mouse RYK protein.

### CROSS-REFERENCE TO RELATED APPLICATIONS

This application claims priority to U.S. Provisional Application No. 63/234,527, filed Aug. 18, 2021, which is hereby incorporated by reference in its entirety and for all purposes.

### STATEMENT AS TO RIGHTS TO INVENTIONS MADE UNDER FEDERALLY SPONSORED RESEARCH AND DEVELOPMENT

This invention was made with government support under CA236361 awarded by the National Institutes of Health. The government has certain rights in the invention.

### SEQUENCE LISTING

The material in the accompanying Sequence Listing is hereby incorporated by reference in its entirety. The accompanying file, named “048537-648001 WO_SL_ST26.xml” was created on Aug. 15, 2022 and is 120,584 bytes. The file can be accessed using Microsoft Word on a computer that uses Windows OS.

### BACKGROUND

Related-to-receptor tyrosine kinase (Ryk) is a highly conserved, single-pass transmembrane receptor for Wingless-int (Wnt) ligands, e.g. Wnt1,¹ Wnt3a,¹ Wnt5a,² and possibly Wnt5b.³ The extracellular domain has a WIF (Wnt inhibitory factor-1 like) domain, which may initiate complex formation with Frizzled (Frz) receptors for crosstalk in the Wnt signaling network to influence activation of β-catenin-dependent and β-catenin-independent (e.g. non-canonical) Wnt signaling pathways.^{1,4,5} The cytoplasmic domain has a tyrosine kinase domain, which lacks apparent kinase activity.⁵⁻⁷. However, the cytoplasmic domain of Ryk may be cleaved by gamma-secretase and released for nuclear translocation,⁸ suggesting that it may play an alternative role in regulating cell signaling.

Ryk apparently primarily plays a role in embryonic development, in which it regulates axonal outgrowth, cardiovascular and craniofacial development, and fetal liver hematopoiesis.^{9-11,12} Genomic disruption of RYK results in perinatal lethality. Despite the importance of Ryk in early development, its expression appears to attenuate during development and does not appear definitively expressed on post-partum tissues. However, because there have not been highly specific anti-Ryk monoclonal antibodies (mAb) of high affinity that react with viable cells expressing Ryk, the post-natal expression of Ryk is understudied.

Of note, however, there are reports describing expression of Ryk by various cancers.⁵ For example, Ryk apparently is expressed in glioblastoma, where it promotes the ‘stemness’ of glioblastoma cells by its capacity to modulate the Wnt/β-catenin pathway.¹³ Moreover, Ryk may be expressed in mammary cancers, where it was purported to promote expansion of breast cancer tumor-initiating cells and shown to enhance mammary cancer cell growth.^{14,15} Ryk also is implicated in gastric cancer tumorgenesis.¹⁶ Again, the evaluation of Ryk expression by various cancers has been handicapped by the lack of highly specific anti-Ryk mAbs with which to examine the relative expression of Ryk on cancer cells and normal post-partum tissues.

There is a need in the art for mAbs that are highly-specific for cells that express Ryk to, for example, study the functional significance of Ryk in early development and in neoplasia. Thus, there is a need in the art for mAbs to target cells that express Ryk for detection, elimination, functional inhibition, immune-mediated destruction, or targeted drug-delivery. Commercial reagents used to assess for expression of Ryk on cells are antibodies derived from heterologous antisera, e.g. sheep anti-Ryk (R&D systems). These anti-Ryk antibodies were selected to have binding for Ryk. However, they are not specific for human Ryk and cross-react with cells that do not express human Ryk. There is one commercially-available mAb that claimed to be specific for human Ryk (e.g. SAP18, LSBio 2401 Fourth Avenue Suite 900, Seattle WA 98121). However, this mAb apparently reacts with denatured human Ryk. As such, this mAb can be used to detect Ryk in immunoblot or ELISA assays, but is not validated for use in flow cytometry because it apparently lacks specific binding for cell-surface Ryk on viable cells. In summary, the previously generated anti-Ryk antibodies do not appear suitable for potential use in the therapy of patients with cancers that express Ryk.

Provided herein, inter alia, are solutions to these and other needs in the art.

### BRIEF SUMMARY

In an aspect is provided an anti-RYK antibody including a heavy chain variable domain and a light chain variable domain, wherein the heavy chain variable domain includes a CDR H1 as set forth in SEQ ID NO:1, a CDR H2 as set forth in SEQ ID NO:2, and a CDR H3 as set forth in SEQ ID NO:3; and wherein the light chain variable domain includes a CDR L1 as set forth in SEQ ID NO: 4, a CDR L2 as set forth in SEQ ID NO:5 and a CDR L3 as set forth in SEQ ID NO:6.

In another aspect is provided an anti-RYK antibody including a heavy chain variable domain and a light chain variable domain, wherein the heavy chain variable domain includes a CDR H1 as set forth in SEQ ID NO: 17, a CDR H2 as set forth in SEQ ID NO: 18, and a CDR H3 as set forth in SEQ ID NO: 19; and wherein the light chain variable domain includes a CDR L1 as set forth in SEQ ID NO:20, a CDR L2 as set forth in SEQ ID NO:21 and a CDR L3 as set forth in SEQ ID NO: 22.

In another aspect is provided an anti-RYK antibody including a heavy chain variable domain and a light chain variable domain, wherein the heavy chain variable domain includes a CDR H1 as set forth in SEQ ID NO:33, a CDR H2 as set forth in SEQ ID NO:34, and a CDR H3 as set forth in SEQ ID NO:35; and wherein the light chain variable domain includes a CDR L1 as set forth in SEQ ID NO:36, a CDR L2 as set forth in SEQ ID NO:37 and a CDR L3 as set forth in SEQ ID NO: 38.

In another aspect is provided an anti-RYK antibody including a heavy chain variable domain and a light chain variable domain, wherein the heavy chain variable domain includes a CDR H1 as set forth in SEQ ID NO:49, a CDR H2 as set forth in SEQ ID NO: 50, and a CDR H3 as set forth in SEQ ID NO:51; and wherein the light chain variable domain includes a CDR L1 as set forth in SEQ ID NO:52, a CDR L2 as set forth in SEQ ID NO:53 and a CDR L3 as set forth in SEQ ID NO: 54.

In another aspect is provided an anti-RYK antibody, wherein the anti-RYK antibody binds the same epitope as an antibody including: a heavy chain variable domain including a CDR H1 as set forth in SEQ ID NO: 1, a CDR H2 as set forth in SEQ ID NO:2, and a CDR H3 as set forth in SEQ ID NO:3, and a light chain variable domain including a CDR L1 as set forth in SEQ ID NO:4, a CDR L2 as set forth in SEQ ID NO:5 and a CDR L3 as set forth in SEQ ID NO:6.

In another aspect is provided an anti-RYK antibody, wherein the anti-RYK antibody binds the same epitope as an antibody including: a heavy chain variable domain including a CDR H1 as set forth in SEQ ID NO: 17, a CDR H2 as set forth in SEQ ID NO: 18, and a CDR H3 as set forth in SEQ ID NO: 19, and a light chain variable domain including a CDR L1 as set forth in SEQ ID NO: 20, a CDR L2 as set forth in SEQ ID NO:21 and a CDR L3 as set forth in SEQ ID NO:22.

In another aspect is provided an anti-RYK antibody, wherein the anti-RYK antibody binds the same epitope as an antibody including: a heavy chain variable domain including a CDR H1 as set forth in SEQ ID NO:33, a CDR H2 as set forth in SEQ ID NO:34, and a CDR H3 as set forth in SEQ ID NO:35, and a light chain variable domain including a CDR L1 as set forth in SEQ ID NO: 36, a CDR L2 as set forth in SEQ ID NO:37 and a CDR L3 as set forth in SEQ ID NO:38.

In another aspect is provided an anti-RYK antibody, wherein the anti-RYK antibody binds the same epitope as an antibody including: a heavy chain variable domain including a CDR H1 as set forth in SEQ ID NO:49, a CDR H2 as set forth in SEQ ID NO:50, and a CDR H3 as set forth in SEQ ID NO:51, and a light chain variable domain including a CDR L1 as set forth in SEQ ID NO: 52, a CDR L2 as set forth in SEQ ID NO:53 and a CDR L3 as set forth in SEQ ID NO:54.

In another aspect is provided an isolated nucleic acid encoding an anti-RYK antibody provided herein including embodiments thereof.

In another aspect is provided a cell including an anti-RYK antibody provided herein including embodiments thereof, or a nucleic acid provided herein including embodiments thereof.

In another aspect is provided a pharmaceutical composition including a therapeutically effective amount of an antibody provided herein including embodiments thereof and a pharmaceutically acceptable excipient.

In another aspect is provided a method of forming an antibody capable of binding to a RYK protein, the method including immunizing a mammal with a peptide including the sequence of SEQ ID NO:129.

In another aspect is provided a method of detecting a RYK-expressing cell, the method including (i) contacting a RYK-expressing cell with an antibody provided herein including embodiments thereof; (ii) and detecting binding of the antibody to a RYK protein expressed by the cell.

In another aspect is provided a method of treating cancer in a subject in need thereof, the method including administering to a subject a therapeutically effective amount of an anti-RYK antibody provided herein including embodiments thereof.

In another aspect is provided a method of identifying an anti-RYK antibody, the method including: (i) contacting an antibody with a first RYK polypeptide including an amino acid sequence corresponding to amino acid residues 48 through 57 of SEQ ID NO:129; (ii) detecting the antibody binding to the first RYK polypeptide; (iii) contacting the antibody with a second RYK polypeptide not including an amino acid sequence corresponding to amino acid residues 48 through 57 of SEQ ID NO: 129; and (iv) detecting the antibody not binding to the second RYK polypeptide, thereby identifying an anti-RYK antibody.

### BRIEF DESCRIPTION OF THE DRAWINGS

FIG. 1 illustrates comparison of the extracellular regions of human and mouse RYK. Alignment of the amino acid sequences of the extracellular region of human (hRYK, upper sequence; SEQ ID NO: 132) and mouse (mRYK, lower sequence, SEQ ID NO:131) RYK are shown. Dots indicate homology at that position, whereas differences are designated by the single letter amino acid codons. The signal peptide and WIF domain are labeled and indicated by lines above the sequence (SEQ ID NO:132).

FIGS. 2A-2D illustrate the amino acid sequence and alignment to the closest mouse IGHV (upper sequence) or IGKV (lower sequence) germline gene depicted for each of the four mouse anti-human RYK hybridomas, designated 2-D11 (FIG. 2A, SEQ ID NO:15 and SEQ ID NO:16), 7-H10 (FIG. 2B, SEQ ID NO:31 and SEQ ID NO:32), 11-E9 (FIG. 2C, SEQ ID NO:47 and SEQ ID NO: 48) and 3-C12 (FIG. 2D, SEQ ID NO:63 and SEQ ID NO:64). For each alignment the upper sequence depicts the amino acid sequence of the heavy or light chain variable region beginning at the first codon of the first framework region and ending with the last codon of the fourth framework region. The lower sequence depicts the amino acid sequence of the heavy or light chain variable region of the most homologous mouse IGHV or IGKV germline gene. Dots indicate homology at that position, whereas differences are designated by the single letter amino acid codons. The framework (FR) and complementarity determining (CDR) regions are marked above the sequences.

FIG. 3 illustrates a comparison of the extracellular domain of hROR1 (SEQ ID NO:129) with mutant forms of hRYK used to map the binding region epitope hRYK bound by each of the anti-human RYK mAbs in this disclosure. The name of the protein represented by the amino acid sequence is on the left margin. Amino acids are indicated by the single-letter amino acid code. The numbers provided on the right margin or above the sequences are the numbers for the position of the amino acid residue below. A dot in the sequence indicates sequence homology with hRYK at that position. A letter indicates the amino acid of the mutant RYK that differs from that present in hRYK at that position. The WIF domain of the RYK extracellular domain is indicated above the amino acid sequence, which is underlined

FIGS. 4A-4B illustrate the denitrification of amino acids required for binding of anti-human RYK mAb to the extracellular domain of human RYK (SEQ ID NO:129). FIG. 4A illustrates experiments in which binding of 2-D11, 7-H10, 3-C12, 11-E9, 6-B5, 6-D10 and sheep anti-RYK mAb was assessed using recombinant human RYK proteins (SEQ ID NO:129) in which one amino acid that differs between human and mouse RYK within the extracellular domain was replaced with the corresponding amino acid of mouse RYK. Each recombinant protein was transferred onto nylon membrane, probed with the indicated anti-RYK mAb or Sheep anti-RYK Ab, and detected with an anti-mouse IgG or donkey-anti-sheep antibody conjugated with horse radish peroxidase. Rabbit anti-IgG blotting is a positive for protein blotting and detection as recombinant proteins have a rabbit IgG tag for purification. Alignment of the protein sequences of the extracellular domain of human and mouse RYK are shown in the lower panel and the boxed amino acids indicate the amino acids changes made for each recombinant protein. FIG. 4B illustrates additional blotting with the 2-D11 antibody to further assess binding, in which substitutions were made within the leader peptide or in the coding region adjacent to the WIF domain of human RYK protein (SEQ ID NO:129) were made. hRYK with mRYK 48-57 is human RYK with the murine amino acids at positions 48-57 substituted, and confers loss of binding to human RYK, as does substitution of the murine leader region fused to human RYK.

FIGS. 5A-5B illustrate variable region sequence of mAb 2-D11. FIG. 5A denotes Ig heavy chain variable region sequence of mAb 2-D11. FIG. 5B denotes Ig kappa chain variable region sequence of mAb 2-D11.

FIGS. 6A-6B illustrate variable region sequence of mAb 7-H10. FIG. 6A denotes Ig heavy chain variable region sequence of mAb 7-H10. FIG. 6B denotes Ig kappa chain variable region sequence of mAb 7-H10.

FIGS. 7A-7B illustrate affinity measurement of binding of the 2-D11 and 7-H10 mAb to recombinant human RYK2 protein. Analysis was performed using a KinExA 3200 instrument. FIG. 7A: The proportion of anti-human RYK mAb bound to particles coated with RYK protein (y-axis) in the presence of increasing molar (M) concentration of soluble RYK competitor (x-axis) is shown for 2-D11 mAb (upper left panel) and 7-H10 mAb (lower left panel). FIG. 7B: Illustration of the 95% confidence interval for the measured K_{D} of 2-D11 mAb (upper right panel) and 7-H10 mAb (lower right panel) for binding to human RYK.

FIG. 8 illustrates the 2-D11 anti-human RYK mAb specifically binds human RYK. Binding of the 2-D11 mAb to human RYK was assessed by flow cytometric staining and analysis of several cell lines. Cells were stained on ice for 20 minutes with 10 μg/ml of 2-D11 anti-human RYK-Alexa647 conjugated mAb (shaded histograms) or equal amounts of isotype matched control mAb (open histograms), washed and analyzed. Histograms depict the relative fluorescence intensity (x axis) of viable cells as determined by light scatter characteristics. ++, + and neg correspond to level of staining as shown in Table 1 for these and other cell lines based on the ratio of median fluorescence intensity (MFI) of stained cells relative to the MFI of isotype control stained cells.

FIG. 9 illustrates examples of 2-D11 staining of lymphoid cells from adult blood, cord blood (N=2), tonsil (N−2), or spleen.

FIG. 10 illustrates primary passage breast cancer patient-derived xenograft (PDX). Primary passage (T1) breast cancer PDX (M0026) derived from an estrogen/progesterone-receptor-negative and HER2-negative breast cancer (triple-negative breast cancer, TNBC). The human TNBC cells of M0026 were dissociated into single cells, which were stained with a fluorochrome-conjugated isotype control mAb (Cont mAb, dark gray histogram) or fluorochrome-conjugated 2-D11 (light gray shaded histogram) and then analyzed on a flow cytometer. The Cont mAb stained cells had the same fluorescence as unstained cells (not shown).

### DETAILED DESCRIPTION

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

### Definitions

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism. In embodiments, the nonspecific nucleic acid sequence does not encode a biological function. In embodiments, the nonspecific nucleic acid sequence is a scrambled nucleic acid sequence. A “scrambled nucleic acid sequence” as provided herein is a recombinant nucleic acid sequence that includes nucleotides randomly linked to each other in vitro. Scrambled nucleic acid sequences are commonly used in the art as control or reference sequences relative to the activity (biological function) of test nucleic acid sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., RYK) in other proteins with different numbering systems. For example, by performing a simple sequence alignment with a protein (e.g., RYK) the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M) (see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2: 482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

“RYK” as referred to herein includes any of the recombinant or naturally-occurring related to receptor tyrosine kinase (RYK) protein or variants or homologs thereof that maintain RYK activity (e.g. within at least 50%, 80%, 90%, 95%, 96%, 97%, 98%, 99% or 100% activity compared to RYK). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring RYK protein. In embodiments, the RYK protein is substantially identical to the protein identified by the UniProt reference number P34925 or a variant or homolog having substantial identity thereto.

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region, involved in binding the target antigen, and a constant (“C”) region that interacts with other components of the immune system. The light and heavy chain variable regions (also referred to herein as light chain variable (VL) domain and heavy chain variable (VH) domain, respectively) come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab₂, bispecific Fab₂, trispecific Fab₃, monovalent IgGs, scFv, bispecific antibodies, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The terms “CDR L1”, “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR H1”, “CDR H2” and “CDR H3” as provided herein refer to the complementarity determining regions (CDR) 1, 2, and 3 of the variable heavy (H) chain of an antibody. In embodiments, the variable heavy chain provided herein includes in N-terminal to C-terminal direction a CDR H1, a CDR H2 and a CDR H3.

The terms “FR L1”, “FR L2”, “FR L3” and “FR L4” as provided herein are used according to their common meaning in the art and refer to the framework regions (FR) 1, 2, 3 and 4 of the variable light (L) chain of an antibody. In embodiments, the variable light chain provided herein includes in N-terminal to C-terminal direction a FR L1, a FR L2, a FR L3 and a FR L4. Likewise, the terms “FR H1”, “FR H2”, “FR H3” and “FR H4” as provided herein are used according to their common meaning in the art and refer to the framework regions (FR) 1, 2, 3 and 4 of the variable heavy (H) chain of an antibody. In embodiments, the variable heavy chain provided herein includes in N-terminal to C-terminal direction a FR H1, a FR H2, a FR H3 and a FR H4.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL), variable light chain (VL) domain or light chain variable region and variable heavy chain (VH), variable heavy chain (VH) domain or heavy chain variable region refer to these light and heavy chain regions, respectively. The terms variable light chain (VL), variable light chain (VL) domain and light chain variable region as referred to herein may be used interchangeably. The terms variable heavy chain (VH), variable heavy chain (VH) domain and heavy chain variable region as referred to herein may be used interchangeably. The Fc (i.e. fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)). The term “antibody” as referred to herein further includes antibody variants such as single domain antibodies. Thus, in embodiments an antibody includes a single monomeric variable antibody domain. Thus, in embodiments, the antibody, includes a variable light chain (VL) domain or a variable heavy chain (VH) domain. In embodiments, the antibody is a variable light chain (VL) domain or a variable heavy chain (VH) domain.

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies (mAb) refer to antibodies derived from a single clone. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31 (3): 169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “ligand” refers to an agent, e.g., a polypeptide or other molecule, capable of binding to a receptor or antibody, antibody variant, antibody region or fragment thereof.

Techniques for conjugating therapeutic agents to antibodies are well known (see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)). As used herein, the term “antibody-drug conjugate” or “ADC” refers to a therapeutic agent conjugated or otherwise covalently bound to an antibody.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetofection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

When the label or detectable moiety is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups. The chelate is normally linked to the PSMA antibody or functional antibody fragment by a group, which enables the formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antibodies and carriers described herein. Macrocyclic chelates such as NOTA, DOTA, and TETA are of use with a variety of metals and radiometals including, but not limited to, radionuclides of gallium, yttrium and copper, respectively. Other ring-type chelates such as macrocyclic polyethers, which are of interest for stably binding nuclides, such as ²²³Ra for RAIT may be used. In certain embodiments, chelating moieties may be used to attach a PET imaging agent, such as an Al-¹⁸F complex, to a targeting molecule for use in PET analysis.

“Contacting” is used in accordance with its plain ordinary meaning and refers to the process of allowing at least two distinct species (e.g. antibodies and antigens) to become sufficiently proximal to react, interact, or physically touch. It should be appreciated; however, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a pharmaceutical composition as provided herein and a cell. In embodiments contacting includes, for example, allowing a pharmaceutical composition as described herein to interact with a cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

As defined herein, the term “inhibition”, “inhibit”, “inhibiting” and the like in reference to cell proliferation (e.g., cancer cell proliferation) means negatively affecting (e.g., decreasing proliferation) or killing the cell. In some embodiments, inhibition refers to reduction of a disease or symptoms of disease (e.g., cancer, cancer cell proliferation). Thus, inhibition includes, at least in part, partially or totally blocking stimulation, decreasing, preventing, or delaying activation, or inactivating, desensitizing, or down-regulating signal transduction or enzymatic activity or the amount of a protein. Similarly an “inhibitor” is a compound or protein that inhibits a receptor or another protein, e.g., by binding, partially or totally blocking, decreasing, preventing, delaying, inactivating, desensitizing, or down-regulating activity (e.g., a receptor activity or a protein activity).

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor.

Thus, the terms “inhibitor,” “repressor” or “antagonist” or “downregulator” interchangeably refer to a substance capable of detectably decreasing the expression or activity of a given gene or protein. The antagonist can decrease protein expression or activity 10%, 20%, 30%, 40%, 50%, 60%, 70%, 80%, 90% or more in comparison to a control in the absence of the antagonist. In certain instances, protein expression or activity is 1.5-fold, 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with the compounds or methods provided herein. The disease may be a cancer. The cancer may refer to a solid tumor malignancy. Solid tumor malignancies include malignant tumors that may be devoid of fluids or cysts. For example, the solid tumor malignancy may include breast cancer, ovarian cancer, pancreatic cancer, cervical cancer, gastric cancer, renal cancer, head and neck cancer, bone cancer, skin cancer or prostate cancer. In some further instances, “cancer” refers to human cancers and carcinomas, sarcomas, adenocarcinomas, lymphomas, leukemias, including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including acute myeloid leukemia (AML), ALL, and CML), or multiple myeloma.

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma (e.g., Mantel cell lymphoma, follicular lymphoma, diffuse large B-cell lymphoma, marginal zona lymphoma, Burkitt's lymphoma), sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia (e.g., lymphoblastic leukemia, chronic lymphocytic leukemia, hairy cell leukemia), acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute myeloid leukemia, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The term “associated” or “associated with” in the context of a substance or substance activity or function associated with a disease (e.g. a protein associated disease, a cancer associated with RKY activity, RKY associated cancer, RKY associated disease (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease. For example, a cancer associated with Ryk activity or function or a RKY associated disease (e.g., cancer, inflammatory disease, autoimmune disease, or infectious disease), may be treated with a RKY modulator or RKY inhibitor, in the instance where increased RKY activity or function (e.g. signaling pathway activity) causes the disease (e.g., cancer, inflammatory disease, autoimmune disease, or infectious disease). For example, an inflammatory disease associated with RKY activity or function or an RKY associated inflammatory disease, may be treated with an RKY modulator or RKY inhibitor, in the instance where increased RKY activity or function (e.g. signaling pathway activity) causes the disease.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

A “therapeutic agent” as referred to herein, is a composition useful in treating or preventing a disease such as cancer (e.g., leukemia). In embodiments, the therapeutic agent is an anti-cancer agent. “Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

An “anticancer agent” as used herein refers to a molecule (e.g. compound, peptide, protein, nucleic acid, 0103) used to treat cancer through destruction or inhibition of cancer cells or tissues. Anticancer agents may be selective for certain cancers or certain tissues. In embodiments, anticancer agents herein may include epigenetic inhibitors and multi-kinase inhibit “Anti-cancer agent” and “anticancer agent” are used in accordance with their plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, melphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossypol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec.®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; or 5-ethynyluracil.

Further examples of anti-cancer agents include, but are not limited to, abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomiphene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; or exemestane.

Further examples of anti-cancer agents include, but are not limited to, fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolidetestrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; or pyridoxylated hemoglobin polyoxyethylerie conjugate.

Further examples of anti-cancer agents include, but are not limited to, raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysins inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine.

Further examples of anti-cancer agents include, but are not limited to, dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin I1 (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol.™ (i.e. paclitaxel), Taxotere.™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), or Altorhyrtins (e.g. Altorhyrtin A and Altorhyrtin C).

Further examples of anti-cancer agents include, but are not limited to Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39·HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin Al (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, Inanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta medica), D-68144 (Asta medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta medica), D-68836 (Asta medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethylstilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

By “therapeutically effective dose or amount” as used herein is meant a dose that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The combined administration contemplates co-administration, using separate formulations or a single pharmaceutical formulation, and consecutive administration in either order, wherein preferably there is a time period while both (or all) active agents simultaneously exert their biological activities.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Anti-Ryk Antibodies

Provided herein are, inter alia, antibodies (e.g., chimeric antibodies, monoclonal antibodies, antibody fragments (e.g., scFvs)) which bind human related-to-tyrosine receptor kinase (RYK) with high efficiency and specificity. The antibodies and antibody compositions provided herein include, for example, novel light and heavy chain domain CDRs and framework regions and are, inter alia, useful for diagnosing and treating cancer and other RYK-related diseases. In embodiments, the anti-RYK antibodies provided herein are capable of binding a human RYK protein, but not a mouse RYK protein.

In an aspect is provided an anti-RYK antibody including a heavy chain variable domain and a light chain variable domain, wherein the heavy chain variable domain includes a CDR H1 as set forth in SEQ ID NO: 1, a CDR H2 as set forth in SEQ ID NO:2, and a CDR H3 as set forth in SEQ ID NO:3; and wherein the light chain variable domain includes a CDR L1 as set forth in SEQ ID NO: 4, a CDR L2 as set forth in SEQ ID NO:5 and a CDR L3 as set forth in SEQ ID NO:6.

In embodiments, the heavy chain variable domain includes the sequence of SEQ ID NO: 15. In embodiments, the heavy chain variable domain is the sequence of SEQ ID NO:15. In embodiments, the light chain variable domain includes the sequence of SEQ ID NO:16. In embodiments, the light chain variable domain is the sequence of SEQ ID NO:16. In embodiments, the heavy chain variable domain includes the sequence of SEQ ID NO:15 and the light chain variable domain includes the sequence of SEQ ID NO:16. In embodiments, the heavy chain variable domain is the sequence of SEQ ID NO: 15 and the light chain variable domain is the sequence of SEQ ID NO:16.

In embodiments, the monoclonal antibody binds RYK with the equilibrium dissociation constants (K_{D}) described in this paragraph. In embodiments, the anti-RYK antibody has a K_{D} of about 2 pM to about 2 nM. In embodiments, the anti-RYK antibody has a K_{D} of about 3 pM to about 2 nM. In embodiments, the anti-RYK antibody has a K_{D} of about 4 pM to about 2 nM. In embodiments, the anti-RYK antibody has a K_{D} of about 5 pM to about 2 nM. In embodiments, the anti-RYK antibody has a K_{D} of about 6 pM to about 2 nM. In embodiments, the anti-RYK antibody has a K_{D} of about 7 pM to about 2 nM. In embodiments, the anti-RYK antibody has a K_{D} of about 8 pM to about 2 nM. In embodiments, the anti-RYK antibody has a K_{D} of about 9 pM to about 2 nM. In embodiments, the anti-RYK antibody has a K_{D} of about 10 pM to about 2 nM. In embodiments, the anti-RYK antibody has a K_{D} of about 50 pM to about 2 nM. In embodiments, the anti-RYK antibody has a K_{D} of about 100 pM to about 2 nM. In embodiments, the anti-RYK antibody has a K_{D} of about 200 pM to about 2 nM. In embodiments, the anti-RYK antibody has a K_{D} of about 300 pM to about 2 nM. In embodiments, the anti-RYK antibody has a K_{D} of about 400 pM to about 2 nM. In embodiments, the anti-RYK antibody has a K_{D} of about 500 pM to about 2 nM. In embodiments, the anti-RYK antibody has a K_{D} of about 600 pM to about 2 nM. In embodiments, the anti-RYK antibody has a K_{D} of about 700 pM to about 2 nM. In embodiments, the anti-RYK antibody has a K_{D} of about 800 pM to about 2 nM. In embodiments, the anti-RYK antibody has a K_{D} of about 900 pM to about 2 nM. In embodiments, the anti-RYK antibody has a K_{D} of about 1 nM to about 2 nM.

In embodiments, the monoclonal antibody binds RYK with the equilibrium dissociation constants (K_{D}) described in this paragraph. In embodiments, the anti-RYK antibody has a K_{D} of about 2 pM to about 1 nM. In embodiments, the anti-RYK antibody has a K_{D} of about 2 pM to about 900 pM. In embodiments, the anti-RYK antibody has a K_{D} of about 2 pM to about 800 pM. In embodiments, the anti-RYK antibody has a K_{D} of about 2 pM to about 700p M. In embodiments, the anti-RYK antibody has a K_{D} of about 2 pM to about 600 pM. In embodiments, the anti-RYK antibody has a K_{D} of about 2 pM to about 500 pM. In embodiments, the anti-RYK antibody has a K_{D} of about 2 pM to about 400 pM. In embodiments, the anti-RYK antibody has a K_{D} of about 2 pM to about 300 pM. In embodiments, the anti-RYK antibody has a K_{D} of about 2 pM to about 200 pM. In embodiments, the anti-RYK antibody has a K_{D} of about 2 pM to about 100 pM. In embodiments, the anti-RYK antibody has a K_{D} of about 2 pM to about 50 pM. In embodiments, the anti-RYK antibody has a K_{D} of about 2 pM to about 10 pM. In embodiments, the anti-RYK antibody has a K_{D} of about 2 pM to about 9 pM. In embodiments, the anti-RYK antibody has a K_{D} of about 2 pM to about 8 pM. In embodiments, the anti-RYK antibody has a K_{D} of about 2 pM to about 7 pM. In embodiments, the anti-RYK antibody has a K_{D} of about 2 pM to about 6 pM. In embodiments, the anti-RYK antibody has a K_{D} of about 2 pM to about 5 pM. In embodiments, the anti-RYK antibody has a K_{D} of about 2 pM to about 4 pM. In embodiments, the anti-RYK antibody has a K_{D} of about 2 pM to about 3 pM.

In embodiments, the monoclonal antibody binds RYK with the equilibrium dissociation constants (K_{D}) described in this paragraph. In embodiments, the anti-RYK antibody has a K_{D} of 2 pM to 2 nM. In embodiments, the anti-RYK antibody has a K_{D} of 3 pM to 2 nM. In embodiments, the anti-RYK antibody has a K_{D} of 4 pM to 2 nM. In embodiments, the anti-RYK antibody has a K_{D} of 5 pM to 2 nM. In embodiments, the anti-RYK antibody has a K_{D} of 6 pM to 2 nM. In embodiments, the anti-RYK antibody has a K_{D} of 7 pM to 2 nM. In embodiments, the anti-RYK antibody has a K_{D} of 8 pM to 2 nM. In embodiments, the anti-RYK antibody has a K_{D} of 9 pM to 2 nM. In embodiments, the anti-RYK antibody has a K_{D} of 10 pM to 2 nM. In embodiments, the anti-RYK antibody has a K_{D} of 50 pM to 2 nM. In embodiments, the anti-RYK antibody has a K_{D} of 100 pM to 2 nM. In embodiments, the anti-RYK antibody has a K_{D} of 200 pM to 2 nM. In embodiments, the anti-RYK antibody has a K_{D} of 300 pM to 2 nM. In embodiments, the anti-RYK antibody has a K_{D} of 400 pM to 2 nM. In embodiments, the anti-RYK antibody has a K_{D} of 500 pM to 2 nM. In embodiments, the anti-RYK antibody has a K_{D} of 600 pM to 2 nM. In embodiments, the anti-RYK antibody has a K_{D} of 700 pM to 2 nM. In embodiments, the anti-RYK antibody has a K_{D} of 800 pM to 2 nM. In embodiments, the anti-RYK antibody has a K_{D} of 900 pM to 2 nM. In embodiments, the anti-RYK antibody has a K_{D} of 1 nM to 2 nM.

In embodiments, the monoclonal antibody binds RYK with the equilibrium dissociation constants (K_{D}) described in this paragraph. In embodiments, the anti-RYK antibody has a K_{D} of 2 pM to 1 nM. In embodiments, the anti-RYK antibody has a K_{D} of 2 pM to 900 pM. In embodiments, the anti-RYK antibody has a K_{D} of 2 pM to 800 pM. In embodiments, the anti-RYK antibody has a K_{D} of 2 pM to 700p M. In embodiments, the anti-RYK antibody has a K_{D} of 2 pM to 600 pM. In embodiments, the anti-RYK antibody has a K_{D} of 2 pM to 500 pM. In embodiments, the anti-RYK antibody has a K_{D} of 2 pM to 400 pM. In embodiments, the anti-RYK antibody has a K_{D} of 2 pM to 300 pM. In embodiments, the anti-RYK antibody has a K_{D} of 2 pM to 200 pM. In embodiments, the anti-RYK antibody has a K_{D} of 2 pM to 100 pM. In embodiments, the anti-RYK antibody has a K_{D} of 2 pM to 50 pM. In embodiments, the anti-RYK antibody has a K_{D} of 2 pM to pM. In embodiments, the anti-RYK antibody has a K_{D} of 2 pM to 9 pM. In embodiments, the anti-RYK antibody has a K_{D} of 2 pM to 8 pM. In embodiments, the anti-RYK antibody has a K_{D} of 2 pM to 7 pM. In embodiments, the anti-RYK antibody has a K_{D} of 2 pM to 6 pM. In embodiments, the anti-RYK antibody has a K_{D} of 2 pM to 5 pM. In embodiments, the anti-RYK antibody has a K_{D} of 2 pM to 4 pM. In embodiments, the anti-RYK antibody has a K_{D} of 2 pM to 3 pM. In embodiments, the anti-RYK antibody has a K_{D} of about 513 pM. In embodiments, the anti-RYK antibody has a K_{D} of 513 pM. In embodiments, the anti-RYK antibody is referred to herein as 2-D11.

In another aspect is provided an anti-RYK antibody including a heavy chain variable domain and a light chain variable domain, wherein the heavy chain variable domain includes a CDR H1 as set forth in SEQ ID NO:17, a CDR H2 as set forth in SEQ ID NO: 18, and a CDR H3 as set forth in SEQ ID NO: 19; and wherein the light chain variable domain includes a CDR L1 as set forth in SEQ ID NO:20, a CDR L2 as set forth in SEQ ID NO:21 and a CDR L3 as set forth in SEQ ID NO: 22.

In embodiments, the heavy chain variable domain includes the sequence of SEQ ID NO: 31. In embodiments, the heavy chain variable domain is the sequence of SEQ ID NO:31. In embodiments, the light chain variable domain includes the sequence of SEQ ID NO:32. In embodiments, the light chain variable domain is the sequence of SEQ ID NO:32. In embodiments, the heavy chain variable domain includes the sequence of SEQ ID NO:31 and the light chain variable domain includes the sequence of SEQ ID NO:32. In embodiments, the heavy chain variable domain is the sequence of SEQ ID NO:31 and the light chain variable domain is the sequence of SEQ ID NO:32.

In embodiments, the monoclonal antibody binds RYK with the equilibrium dissociation constants (K_{D}) described in this paragraph. In embodiments, the anti-RYK antibody has a K_{D} of about 6 nM to about 17 nM. In embodiments, the anti-RYK antibody has a K_{D} of about 7 nM to about 17 nM. In embodiments, the anti-RYK antibody has a K_{D} of about 8 nM to about 17 nM. In embodiments, the anti-RYK antibody has a K_{D} of about 9 nM to about 17 nM. In embodiments, the anti-RYK antibody has a K_{D} of about 10 nM to about 17 nM. In embodiments, the anti-RYK antibody has a K_{D} of about 11 nM to about 17 nM. In embodiments, the anti-RYK antibody has a K_{D} of about 12 nM to about 17 nM. In embodiments, the anti-RYK antibody has a K_{D} of about 13 nM to about 17 nM. In embodiments, the anti-RYK antibody has a K_{D} of about 14 nM to about 17 nM. In embodiments, the anti-RYK antibody has a K_{D} of about 15 nM to about 17 nM. In embodiments, the anti-RYK antibody has a K_{D} of about 16 nM to about 17 nM.

In embodiments, the monoclonal antibody binds RYK with the equilibrium dissociation constants (K_{D}) described in this paragraph. In embodiments, the anti-RYK antibody has a K_{D} of 6 nM to 16 nM. In embodiments, the anti-RYK antibody has a K_{D} of about 6 nM to about 15 nM. In embodiments, the anti-RYK antibody has a K_{D} of about 6 nM to about 14 nM. In embodiments, the anti-RYK antibody has a K_{D} of about 6 nM to about 13 nM. In embodiments, the anti-RYK antibody has a K_{D} of about 6 nM to about 12 nM. In embodiments, the anti-RYK antibody has a K_{D} of about 6 nM to about 11 nM. In embodiments, the anti-RYK antibody has a K_{D} of about 6 nM to about 10 nM. In embodiments, the anti-RYK antibody has a K_{D} of about 6 nM to about 9 nM. In embodiments, the anti-RYK antibody has a K_{D} of about 6 nM to about 8 nM. In embodiments, the anti-RYK antibody has a K_{D} of about 6 nM to about 7 nM.

In embodiments, the monoclonal antibody binds RYK with the equilibrium dissociation constants (K_{D}) described in this paragraph. In embodiments, the anti-RYK antibody has a K_{D} of 6 nM to 17 nM. In embodiments, the anti-RYK antibody has a K_{D} of 7 nM to 17 nM. In embodiments, the anti-RYK antibody has a K_{D} of 8 nM to 17 nM. In embodiments, the anti-RYK antibody has a K_{D} of 9 nM to 17 nM. In embodiments, the anti-RYK antibody has a K_{D} of 10 nM to 17 nM. In embodiments, the anti-RYK antibody has a K_{D} of 11 nM to 17 nM. In embodiments, the anti-RYK antibody has a K_{D} of 12 nM to 17 nM. In embodiments, the anti-RYK antibody has a K_{D} of 13 nM to 17 nM. In embodiments, the anti-RYK antibody has a K_{D} of about 14 nM to about 17 nM. In embodiments, the anti-RYK antibody has a K_{D} of 15 nM to 17 nM. In embodiments, the anti-RYK antibody has a K_{D} of 16 nM to 17 nM.

In embodiments, the monoclonal antibody binds RYK with the equilibrium dissociation constants (K_{D}) described in this paragraph. In embodiments, the anti-RYK antibody has a K_{D} of 6 nM to 16 nM. In embodiments, the anti-RYK antibody has a K_{D} of 6 nM to 15 nM. In embodiments, the anti-RYK antibody has a K_{D} of 6 nM to 14 nM. In embodiments, the anti-RYK antibody has a K_{D} of 6 nM to 13 nM. In embodiments, the anti-RYK antibody has a K_{D} of 6 nM to 12 nM. In embodiments, the anti-RYK antibody has a K_{D} of 6 nM to about 11 nM. In embodiments, the anti-RYK antibody has a K_{D} of 6 nM to 10 nM. In embodiments, the anti-RYK antibody has a K_{D} of 6 nM to 9 nM. In embodiments, the anti-RYK antibody has a K_{D} of 6 nM to 8 nM. In embodiments, the anti-RYK antibody has a K_{D} of abut 6 nM to 7 nM. In embodiments, the anti-RYK antibody has a K_{D} of about 10 nM. In embodiments, the anti-RYK antibody has a K_{D} of 10 nM. In embodiments, the anti-RYK antibody is referred to herein as 7-D10.

In another aspect is provided an anti-RYK antibody including a heavy chain variable domain and a light chain variable domain, wherein the heavy chain variable domain includes a CDR H1 as set forth in SEQ ID NO:33, a CDR H2 as set forth in SEQ ID NO: 34, and a CDR H3 as set forth in SEQ ID NO:35; and wherein the light chain variable domain includes a CDR L1 as set forth in SEQ ID NO:36, a CDR L2 as set forth in SEQ ID NO:37 and a CDR L3 as set forth in SEQ ID NO: 38.

In embodiments, the heavy chain variable domain includes the sequence of SEQ ID NO: 47. In embodiments, the heavy chain variable domain is the sequence of SEQ ID NO:47. In embodiments, the light chain variable domain includes the sequence of SEQ ID NO:48. In embodiments, the light chain variable domain is the sequence of SEQ ID NO:48. In embodiments, the heavy chain variable domain includes the sequence of SEQ ID NO:47 and the light chain variable domain includes the sequence of SEQ ID NO:48. In embodiments, the heavy chain variable domain is the sequence of SEQ ID NO:47 and the light chain variable domain is the sequence of SEQ ID NO:48. In embodiments, the anti-RYK antibody is referred to herein as 11-E9.

In another aspect is provided an anti-RYK antibody including a heavy chain variable domain and a light chain variable domain, wherein the heavy chain variable domain includes a CDR H1 as set forth in SEQ ID NO:49, a CDR H2 as set forth in SEQ ID NO: 50, and a CDR H3 as set forth in SEQ ID NO:51; and wherein the light chain variable domain includes a CDR L1 as set forth in SEQ ID NO:52, a CDR L2 as set forth in SEQ ID NO:53 and a CDR L3 as set forth in SEQ ID NO: 54.

In embodiments, the heavy chain variable domain includes the sequence of SEQ ID NO: 63. In embodiments, the heavy chain variable domain is the sequence of SEQ ID NO:63. In embodiments, the light chain variable domain includes the sequence of SEQ ID NO:64. In embodiments, the light chain variable domain is the sequence of SEQ ID NO:64. In embodiments, the heavy chain variable domain includes the sequence of SEQ ID NO:63 and the light chain variable domain includes the sequence of SEQ ID NO:64. In embodiments, the heavy chain variable domain is the sequence of SEQ ID NO:63 and the light chain variable domain is the sequence of SEQ ID NO:64. In embodiments, the anti-RYK antibody is referred to herein as 3-C12.

In embodiments, the anti-RYK antibody is a chimeric antibody. In embodiments, the anti-RYK antibody is a Fab' fragment. In embodiments, the anti-RYK antibody is an IgG. In embodiments, the light chain variable domain and said heavy chain variable domain form part of a scFv.

In embodiments, the anti-RYK antibody is capable of binding a RYK protein. In embodiments, the anti-RYK antibody binds an extracellular RYK domain. In embodiments, the anti-RYK antibody binds a human extracellular RYK domain. In embodiments, the anti-RYK antibody binds an extracellular RYK domain including the amino acid sequence of SEQ ID NO: 129. In embodiments, the anti-RYK antibody binds an extracellular RYK domain that is the amino acid sequence of SEQ ID NO:129. In embodiments, the anti-RYK antibody binds an amino acid sequence corresponding to amino acid residues 48 through 57 of SEQ ID NO:129. In embodiments, the anti-RYK antibody is bound to a RYK protein. In embodiments, the RYK protein is a human RYK protein. In embodiments, the RYK protein includes the sequence of SEQ ID NO:130. In embodiments, the RYK protein is the sequence of SEQ ID NO: 130. In embodiments, the RYK protein is the sequence of SEQ ID NO: 129. In embodiments, the RYK protein does not bind to a mouse RYK protein. In embodiments, the anti-RYK antibody does not bind a RYK protein including an amino acid sequence corresponding to amino acid residues 32 through 41 of SEQ ID NO: 131. In embodiments, the anti-RYK antibody does not bind a RYK protein including the sequence of SEQ ID NO:131. In embodiments, the anti-RYK antibody does not bind a RYK protein of SEQ ID NO: 131. In embodiments, the anti-RYK antibody does not bind a mouse extracellular RYK domain. In embodiments, the RYK protein forms part of a cell. In embodiments, the RYK protein is expressed on the surface of a cell.

In another aspect is provided an anti-RYK antibody, wherein the anti-RYK antibody binds the same epitope as an antibody including: a heavy chain variable domain including a CDR H1 as set forth in SEQ ID NO: 1, a CDR H2 as set forth in SEQ ID NO:2, and a CDR H3 as set forth in SEQ ID NO:3, and a light chain variable domain including a CDR L1 as set forth in SEQ ID NO:4, a CDR L2 as set forth in SEQ ID NO:5 and a CDR L3 as set forth in SEQ ID NO:6.

In another aspect is provided an anti-RYK antibody, wherein the anti-RYK antibody binds the same epitope as an antibody including: a heavy chain variable domain including a CDR H1 as set forth in SEQ ID NO:17, a CDR H2 as set forth in SEQ ID NO: 18, and a CDR H3 as set forth in SEQ ID NO: 19, and a light chain variable domain including a CDR L1 as set forth in SEQ ID NO: 20, a CDR L2 as set forth in SEQ ID NO:21 and a CDR L3 as set forth in SEQ ID NO:22.

In another aspect is provided an anti-RYK antibody, wherein the anti-RYK antibody binds the same epitope as an antibody including: a heavy chain variable domain including a CDR H1 as set forth in SEQ ID NO:33, a CDR H2 as set forth in SEQ ID NO:34, and a CDR H3 as set forth in SEQ ID NO:35, and a light chain variable domain including a CDR L1 as set forth in SEQ ID NO: 36, a CDR L2 as set forth in SEQ ID NO:37 and a CDR L3 as set forth in SEQ ID NO:38.

In another aspect is provided an anti-RYK antibody, wherein said anti-RYK antibody binds the same epitope as an antibody including: a heavy chain variable domain including a CDR H1 as set forth in SEQ ID NO:49, a CDR H2 as set forth in SEQ ID NO: 50, and a CDR H3 as set forth in SEQ ID NO:51, and a light chain variable domain including a CDR L1 as set forth in SEQ ID NO: 52, a CDR L2 as set forth in SEQ ID NO:53 and a CDR L3 as set forth in SEQ ID NO:54.

In embodiments, the anti-RYK antibody is attached to a therapeutic or a diagnostic moiety. In embodiments, the anti-RYK antibody is attached to a therapeutic moiety. In embodiments, the anti-RYK antibody is attached to a diagnostic moiety.

### Nucleic Acid Compositions

The compositions provided herein include nucleic acid molecules encoding the anti-RYK antibodies or portions thereof provided herein including embodiments thereof. The antibodies encoded by the isolated nucleic acid provided herein are described in detail throughout this application (including the description above and in the examples section). Thus, in an aspect is provided an isolated nucleic acid encoding an anti-RYK antibody provided herein including embodiments thereof.

### Antibody Compositions

The light and heavy chains of the antibodies provided herein may, inter alia, form part of recombinant proteins (e.g., chimeric antigen receptors (CARs) or bispecific antibodies (BiTes) using conventional methods well known in the art. Through the recruitment of effector cells, the anti-RYK antibodies provided herein may induce cell killing of RYK-expressing cells and therefore be useful for therapeutic purposes when used by themselves or in the context of a CAR or BiTe.

### Cell Compositions

The compositions provided herein include cell compositions including the anti-RYK antibodies provided herein including embodiments thereof. Thus, in an aspect is provided a cell comprising an anti-RYK antibody provided herein including embodiments thereof, or a nucleic acid provided herein including embodiments thereof.

### Pharmaceutical Compositions

The compositions provided herein include pharmaceutical compositions including the anti-RYK antibodies provided herein including embodiments thereof. Thus, in an aspect is provided a pharmaceutical composition comprising a therapeutically effective amount of an antibody provided herein including embodiments thereof and a pharmaceutically acceptable excipient.

### Methods

In an aspect is provided a method of forming an antibody capable of binding to a RYK protein, said method including immunizing a mammal with a peptide including the sequence of SEQ ID NO: 129.

In another aspect is provided a method of detecting a RYK-expressing cell, the method including (i) contacting a RYK-expressing cell with an antibody provided herein including embodiments thereof; (ii) and detecting binding of the antibody to a RYK protein expressed by the cell.

In embodiments, the antibody is attached to a detectable moiety. In embodiments, the biological sample is whole blood, blood fractions or products, tissue, or cultured cells. In embodiments, the biological sample is whole blood. In embodiments, the biological sample is blood fractions or products. In embodiments, the biological sample is blood fractions. In embodiments, the biological sample is blood products. In embodiments, the biological sample is tissue. In embodiments, the biological sample is cultured cells.

In embodiments, the RYK-expressing cell is a cancer cell. In embodiments, the cancer cell is a bladder cancer cell, a brain cancer cell, a breast cancer cell, a chronic myeloid leukemia (CML) cell, a colon cancer cell, an Ewing's sarcoma cell, a lung cancer cell, a mantle cell lymphoma cell, an ovarian cancer cell, a pancreas cancer cell, a skin cancer cell or a melanoma cell. In embodiments, the cancer cell is a bladder cancer cell. In embodiments, the cancer cell is a brain cancer cell. In embodiments, the cancer cell is a breast cancer cell. In embodiments, the cancer cell is a chronic myeloid leukemia (CML) cell. In embodiments, the cancer cell is a colon cancer cell. In embodiments, the cancer cell is an Ewing's sarcoma cell. In embodiments, the cancer cell is a lung cancer cell. In embodiments, the cancer cell is a mantle cell lymphoma cell. In embodiments, the cancer cell is an ovarian cancer cell. In embodiments, the cancer cell is a pancreas cancer cell. In embodiments, the cancer cell is a skin cancer cell. In embodiments, the cancer cell is a melanoma cell.

In another aspect is provided a method of treating cancer in a subject in need thereof, the method including administering to a subject a therapeutically effective amount of an anti-RYK antibody provided herein including embodiments thereof.

In embodiments, the cancer is bladder cancer, brain cancer, breast cancer, chronic myeloid leukemia (CML), colon cancer, Ewing's sarcoma, lung cancer, mantle cell lymphoma, ovarian cancer, pancreas cancer, skin cancer, or melanoma. In embodiments, the cancer is bladder cancer. In embodiments, the cancer is brain cancer. In embodiments, the cancer is chronic myeloid leukemia (CML). In embodiments, the cancer is colon cancer. In embodiments, the cancer is Ewing's sarcoma. In embodiments, the cancer is lung cancer. In embodiments, the cancer is mantle cell lymphoma. In embodiments, the cancer is ovarian cancer. In embodiments, the cancer is pancreas cancer. In embodiments, the cancer is skin cancer. In embodiments, the cancer is melanoma.

In another aspect is provided a method of identifying an anti-RYK antibody, the method including: (i) contacting an antibody with a first RYK polypeptide including an amino acid sequence corresponding to amino acid residues 48 through 57 of SEQ ID NO:129; (ii) detecting the antibody binding to the first RYK polypeptide; (iii) contacting the antibody with a second RYK polypeptide not including an amino acid sequence corresponding to amino acid residues 48 through 57 of SEQ ID NO: 129; and (iv) detecting the antibody not binding to the second RYK polypeptide, thereby identifying an anti-RYK antibody.

In embodiments, the antibody is a chimeric antibody. In embodiments the antibody is a Fab' fragment. In embodiments, the antibody is a single chain antibody.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1

Applicants have generated mAbs that are highly specific for the extracellular domain of human Ryk, but that do not bind to highly homologous mouse Ryk. These mAbs apparent bind to an epitope(s) found at the amino terminus of the mature human Ryk protein. Moreover, these mAbs have very high affinity for Ryk and surprisingly do not appear to react with normal human post-partum tissues. For example, Applicants found that these mAbs do not bind hematopoietic cells in normal human cord blood or post-partum blood, tonsil, spleen, or bone marrow. However, surprisingly these mAbs react with cancer cell lines derived from a variety of different solid tumors or blood cancers. Because these mAb do not react with all cancers derived from the same tissue type, we surmise that these mAbs do not react with all post-partum cells derived from that tissue type. Surprisingly, Applicants discovered that the mAbs react very strongly with primary tumor cells of patients with aggressive cancers, such as triple-negative, metastatic breast cancer cells. Moreover, Applicants have found that these mAb react strongly with cancer cells in malignant pleural effusions or ascites, and may react with circulating tumor cells in the blood. Preliminary studies also indicate that these mAbs react most strongly with cancer cells that have cancer-stem-cell or mesenchymal features, which are associated with a high metastatic trajectory and poor prognosis.

These antibodies can be used to bind cancer cells that express Ryk. Because Ryk apparently has low-to-negligible expression on normal post-partum tissues, these mAbs may target Ryk-expressing cancer cells for destruction, either via antibody-dependent cellular cytotoxicity (ADCC), or by inhibiting the yet-to-be-defined function of Ryk, which apparently promotes cancer cell migration, growth, and/or cancer-stem cell renewal. In addition, these mAbs may be linked with toxins, allowing for specific delivery of an anti-Ryk mAb-bound toxin to tumor cells that express Ryk. Because of their high affinity, they can be ‘humanized’ to assimilate human antibodies and/or used to generate single-chain-Fv (scFv) domains that bind to Ryk, which could be used to fashion bi-specific antibodies targeting Ryk along with another molecule, such as CD3, which could allow for the cellular immune activation of an anti-tumor immune response against Ryk-expressing tumor cells. Moreover, these anti-Ryk scFv could be used to generate chimeric antigen receptors (CARs). Expression of anti-Ryk CARs by T cells or NK cells could allow for anti-Ryk CAR T/NK cell therapy directed against Ryk-expressing cancers.

As described herein, Applicants have made anti-Ryk antibodies and have measured their affinities to antigen. Further, the Ryk binding site has been sequenced and the amino acid sequences of the heavy and light chain of two disclosed anti-Ryk mAb, 2-D11 and 7-H10 (forthcoming) were determined. The antibodies have been tested in cell lines of various cancers, including xenografts of triple negative breast cancer. Further, the 2-D11 antibody has been used to stain cancer cell lines derived from various cancer tissue types, in including primary tumor cells from breast cancer patient-derived xenografts (e.g. a triple-negative metastatic breast cancer).

### Example 3

Anti-RYK mAb were generated for binding to leukemia cells of patients with chronic lymphocytic leukemia (CLL), since data suggests that RYK was expressed on CLL cells in studies using anti-RYK antisera. However, upon generating high-affinity mAbs specific for RYK, Applicants were surprised to find that the anti-RYK antisera apparently had spurious binding activity for CLL cells, which were found to actually not express RYK and do not react with the disclosed anti-Ryk mAbs described herein. Applicants also found that RYK is not expressed on all non-cancer cells tested, but is expressed on the neoplastic cells of many different human cancers.

### Example 3

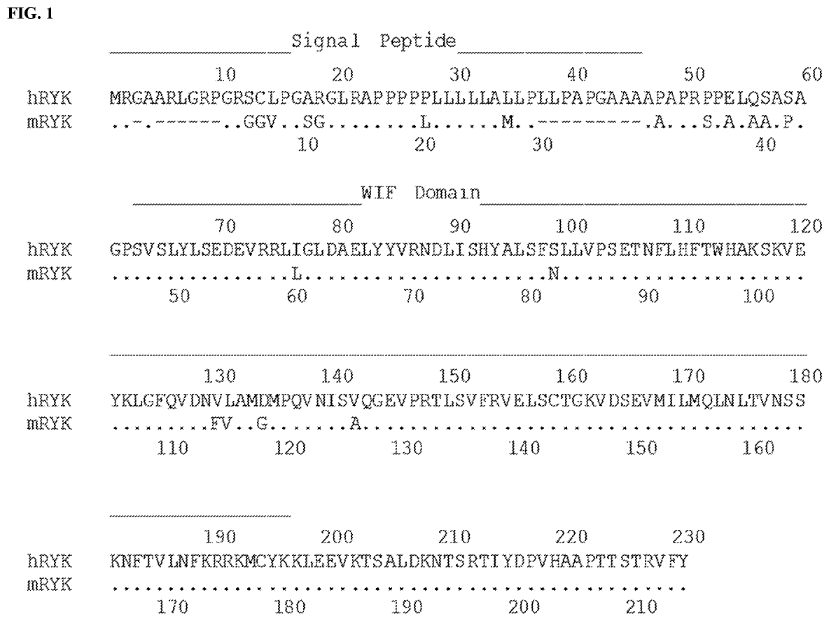

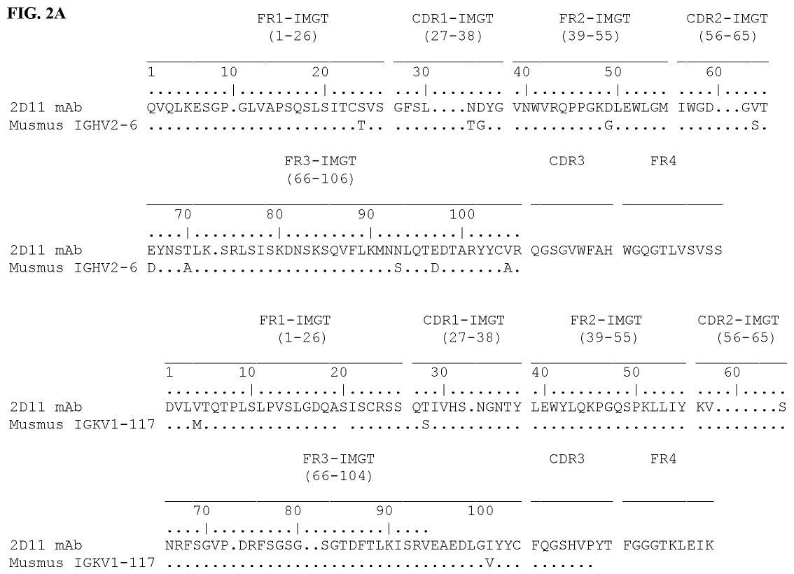

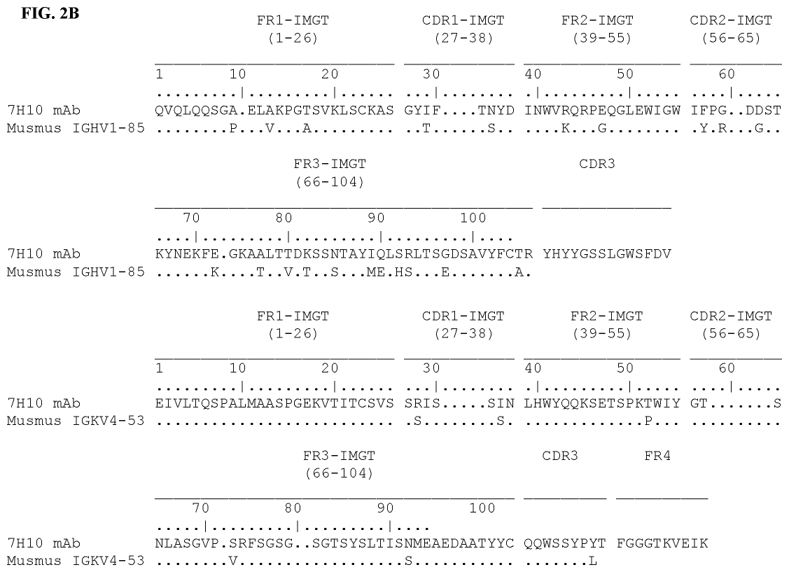

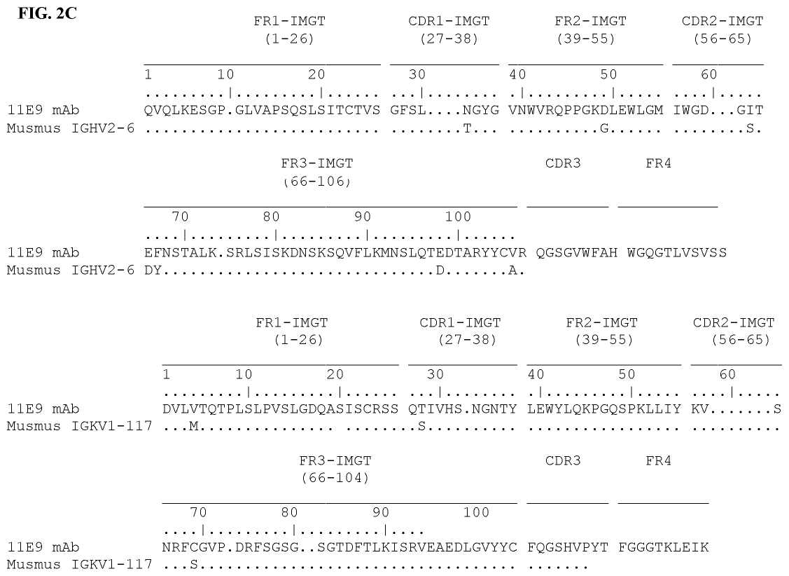

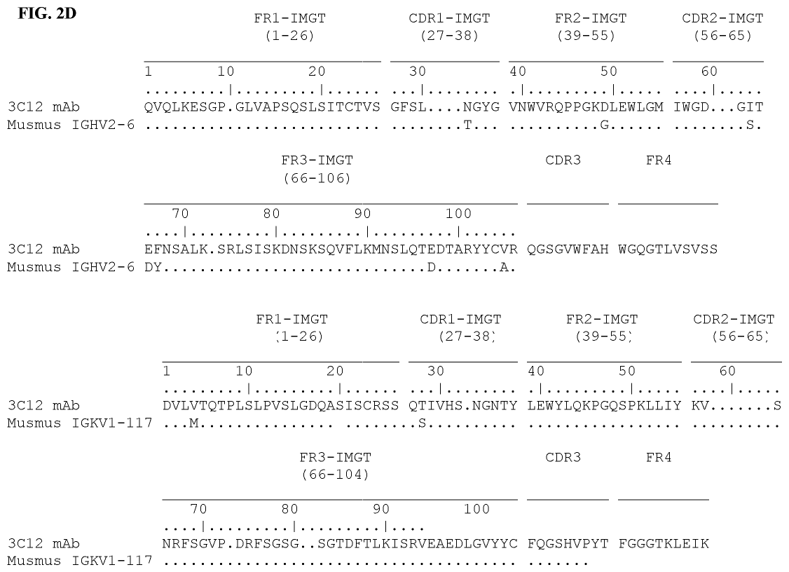

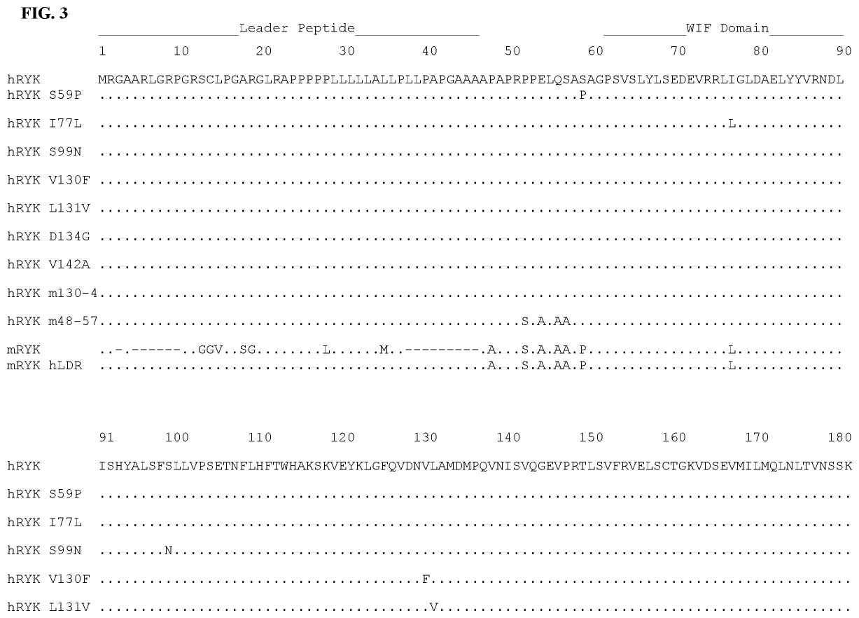

The extracellular protein sequence of human Ryk is highly homologous with the extracellular protein sequence of mouse RYK (FIG. 1). Anti-human RYK mAbs that bind the mature extracellular protein domain of human RYK, consisting of the amino acids shown in FIG. 2, but that do not bind the highly homologous mouse RYK, consisting of amino acids shown in FIG. 3, have been generated.

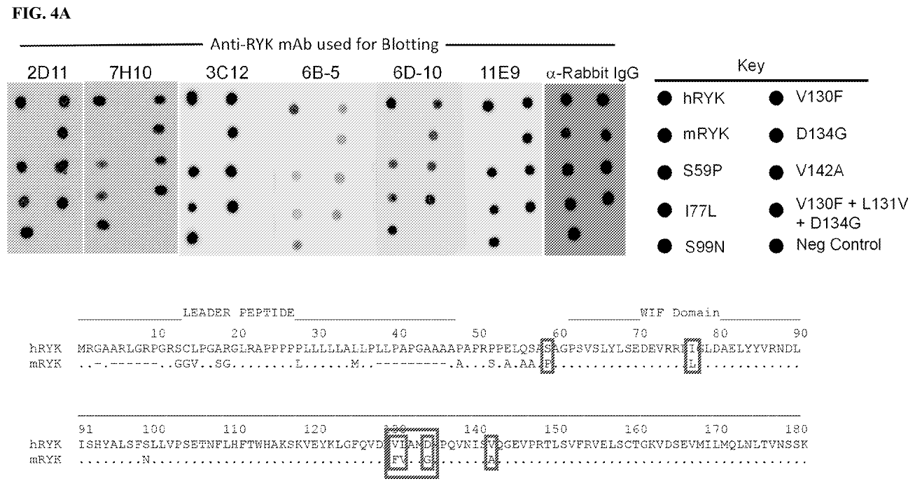

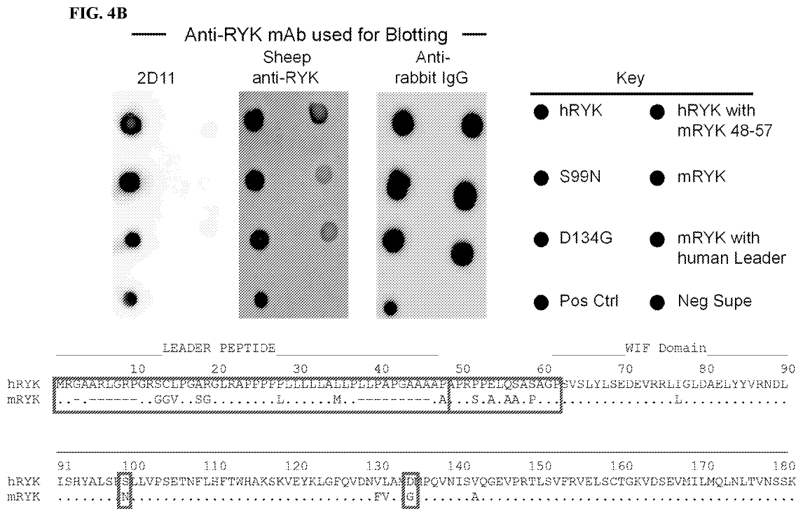

The anti-human RYK mAbs, 2-D11 and 7-H10, appear specific for an amino terminal epitope of human Ryk (residues 46-57) that are distinct from those in mouse Ryk (FIG. 3); these mAbs also bind mutant forms of Ryk containing amino acid substitutions in human Ryk at positions that differ from that of the highly homologous mouse Ryk to assimilate mouse Ryk at the site of substitution, with amino acid sequence shown in FIG. 3, as shown by immunoblot analyses of recombinant extracellular proteins of human or mouse or each of the various mutant forms of human Ryk (FIG. 4) For this, the binding of 2-D11, 7-H10, or Sheep anti-RYK mAb was assessed using recombinant extracellular RYK proteins as specified in the Key. Each recombinant protein was transferred onto nylon membrane, probed with the 2-D11, 7-H10, or Sheep anti-RYK, and detected with an anti-mouse IgG or donkey-anti-sheep antibody conjugated with horse radish peroxidase. A black dot represents a positive signal for antibody binding. As seen in FIG. 4, the sheep anti-Ryk reacted with either human or mouse Ryk, whereas 2-D11 or 7-H10 each reacted with human Ryk, but not mouse Ryk. Moreover, 2-D11 or 7-H10 reacted with each of the mutant forms of human Ryk harboring amino acid substitution(s) at specific sites at which the human Ryk sequence differed from the mouse Ryk sequence; the amino acid sequences of each of these recombinant proteins are provided in FIG. 3.

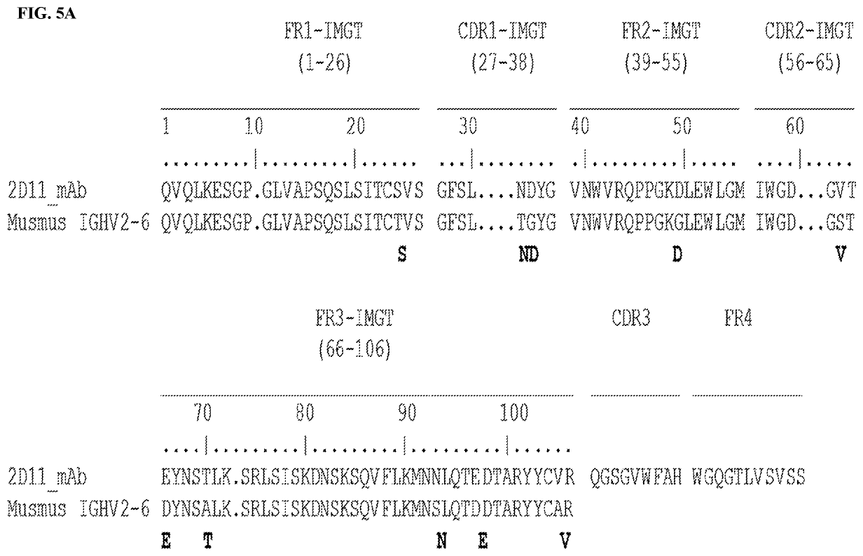

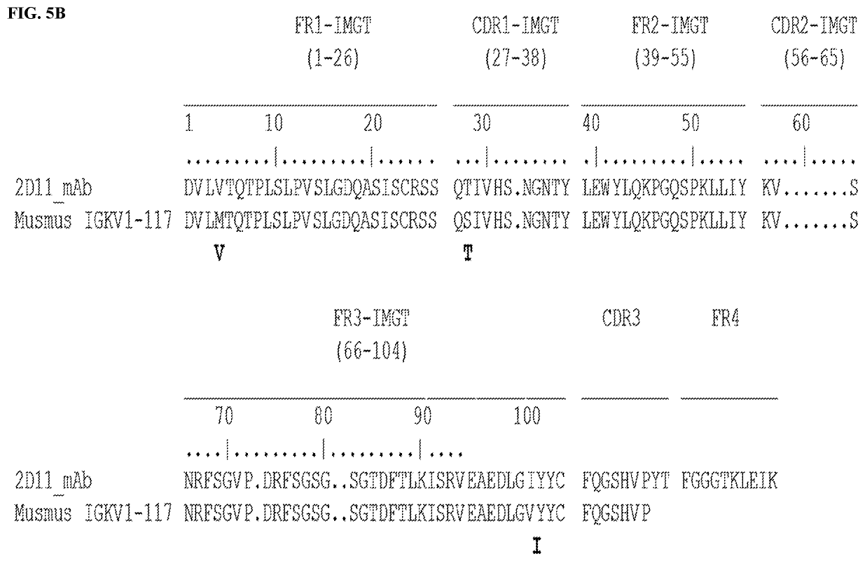

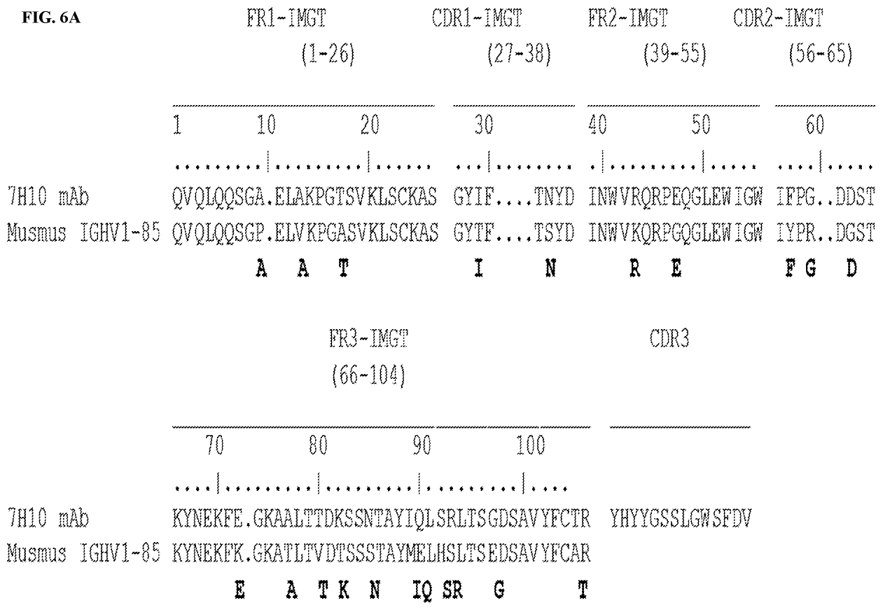

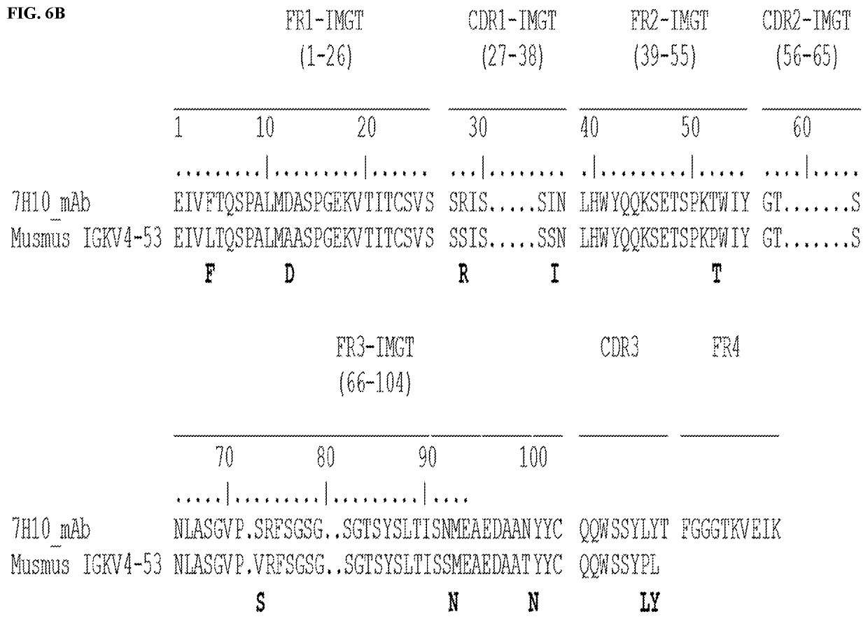

The mAb 2-D11, referenced above, has the heavy chain variable region sequence shown in FIG. 5A and the light chain variable region sequence of 2-D11 is provided in FIG. 5B. The mAb 7-H10, referenced above, has the heavy chain variable region sequence shown in FIG. 6A and the light chain variable region sequence of 2-D11 is provided in FIG. 6B. Also presented in these figures is the mouse germline heavy chain variable region gene (FIG. 5A, FIG. 6A) or mouse germline light chain variable region gene (FIG. 5B, FIG. 6B) that is closest in sequence homology to 2-D11 or 7-H10, respectively.

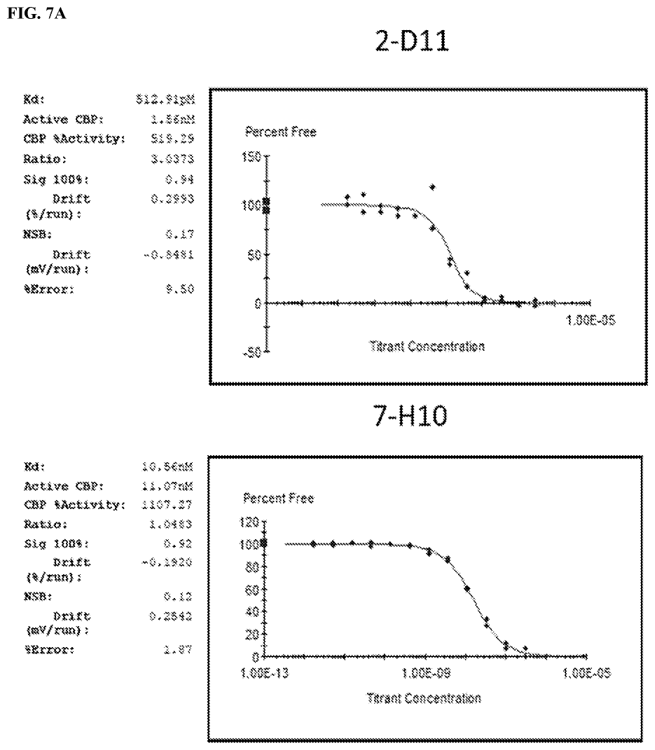

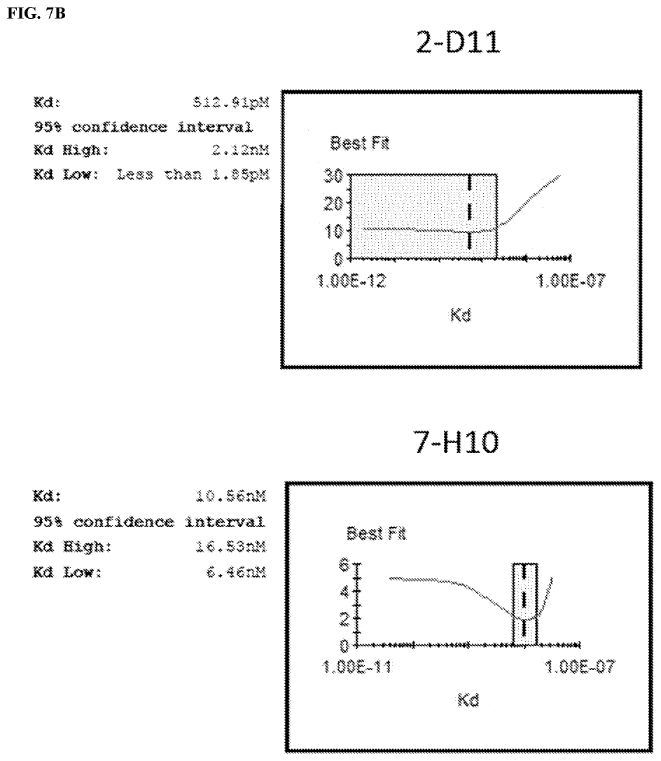

The KinExA binding data of 2-D11 or 7-H10 for human Ryk is provided in FIG. 7. The calculated Kd of 2-D11 for human Ryk is 512.9 pM; the calculated Kd of 7-H10 for human Ryk is 10.56 nM.

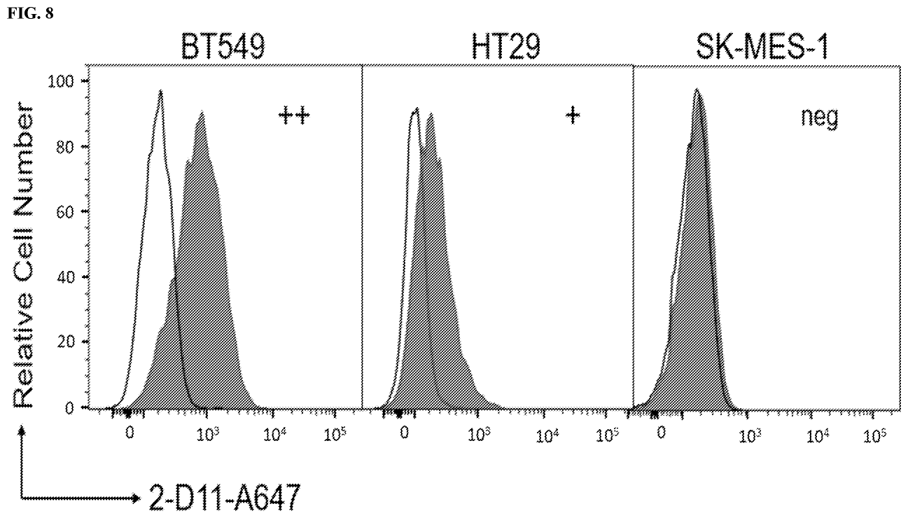

The 2-D11 mAb was conjugated with a fluorochrome (Alexa 647) and the conjugated mAb was used to stain established tumor cell lines. The open histograms depict the fluorescence intensity of cell lines stained with a control fluorochrome-conjugated mAb of irrelevant specificity (FIG. 8). The shaded histograms depict the fluorescence of cells stained with the 2-D11-fluorochrome-conjugated mAb. Representative staining of tumor cell lines are provided, showing “++” staining (for BT549), “+” staining (for HT29), or no staining or “neg” (for SK-MES-1) relative to that of cells treated with a fluorochrome-conjugated ‘control mAb’ of irrelevant specificity that does not bind these cells. These control-mAb-stained cells had a fluorescence that was the same as that of unstained cells.

Table 1 provides the flow cytometry data on various cancer cell lines stained with fluorochrome-conjugated 2-D11, as depicted in FIG. 8. The name of each cell line is listed in the first column and the tissue of origin is listed in the second column. Binding of the 2-D11 mAb to human RYK was assessed by flow cytometric staining and analysis by staining on ice for 20 minutes with 5 μg/ml of 2-D11 anti-human RYK-Alexa647 conjugated mAb or equal amounts of isotype matched control mAb, washed, and analyzed. The median fluorescence intensity (MFI) of 2-D11 stained cells is listed in the middle column and the MFI of isotype control stained cells are listed in the in the adjacent column. Cell lines were scored as “++”, “+”, or neg (unmarked with “+”) lines based on the ratio of median fluorescence intensity (MFI) of stained cells relative to the MFI of isotype control stained cells (MFIR).

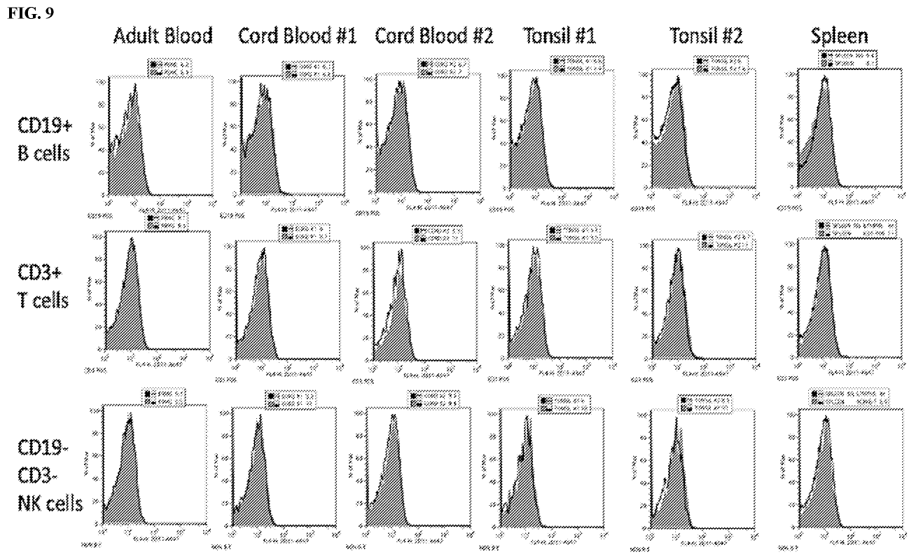

FIG. 9 provides flow cytometry data on fluorochrome-conjugated 2-D11 staining of lymphoid cells found in adult blood, cord blood (N=2), tonsil (N=2), or spleen, as indicated in the top of each column. The open histograms represent cells stained with a fluorochrome-conjugated mAb of irrelevant specificity (control staining) in lieu of 2-D11. The shaded histograms are of cells stained with 2-D11. The cells also were stained with a mAb conjugated with fluorochromes of different colors that were specific for CD19 or CD3. The top row provides data on gated CD19+B cells, the middle row provides data on gated CD3+ T cells, and the bottom row provide data on cells that lack binding to mAbs specific for CD19 or CD3 (NK cells). As seen from these data, 2-D11 does not react with normal human lymphoid cells.

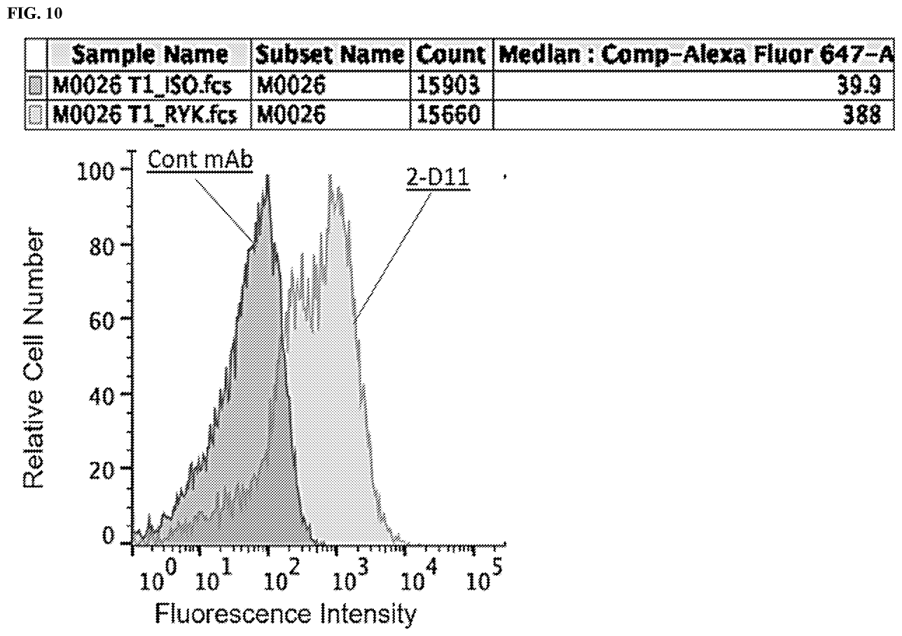

FIG. 10 shows representative staining of 2-D11 for primary human breast cancer cells. The cells stained in FIG. 10 were dissociated single cells prepared from a patient-derived xenograft (PDX), which we generated by engrafting immune-deficient with triple-negative (ER/PR-, HER2-) breast adenocarcinoma tissue removed from a patient with metastatic breast cancer.

### Tables

Table 1. Flow cytometric staining analysis of cancer cell lines with anti-human RYK mAb.

The name of each cell line is listed in the first column and the tissue of origin is listed in the second column. Binding of the 2-D11 mAb to human RYK was assessed by flow cytometric staining and analysis by staining on ice for 20 minutes with 5 μg/ml of 2-D11 anti-human RYK-Alexa647 conjugated mAb or equal amounts of isotype matched control mAb, washed and analyzed. The median fluorescence intensity (MFI) of 2-D11 stained cells is listed in the middle column and the MFI of isotype control stained cells are listed in the in the adjacent column. Cell lines were scored as “++”, “+” or neg (unmarked with “+”) lines based on the ratio of median fluorescence intensity (MFI) of stained cells relative to the MFI of isotype control stained cells (MFIR).

| NAME | TISSUE | 2011 MFI | Isotope MFI | 2-D11 MFIR |
|---|---|---|---|---|
| NALM6 | ALL* | 7.3 | 5.7 | 1.3 |
| REH | ALL* | 7.6 | 7.4 | 1.0 |
| T24 + | BLADDER | 119 | 72.6 | 1.6 |
| HS683 ++ | BRAIN | 576 | 229 | 2.5 |
| SH-SY5Y + | BRAIN | 8.1 | 4.1 | 2.0 |
| BT549 ++ | BREAST | 777 | 197 | 3.9 |
| HS-578T ++ | BREAST | 20 | 7.3 | 2.7 |
| T47-D + | BREAST | 50.9 | 25.7 | 2.0 |
| SKBR3 + | BREAST | 117 | 73.9 | 1.6 |
| MCF7 | BREAST | 7.4 | 5.2 | 1.4 |
| MDA-MB-231 | BREAST | 9.2 | 8.3 | 1.1 |
| RAMOS | BURKITT NHL* | 6.5 | 6.1 | 1.1 |
| RAJI | BURKITT NHL* | 6.6 | 6.4 | 1.0 |
| MEC1 | CLL* | 4.8 | 4.3 | 1.1 |
| K562 + | CML* | 4.2 | 2.8 | 1.5 |
| HT29 ++ | COLON | 224 | 100 | 2.2 |
| SW620 | COLON | 6.5 | 5.6 | 1.2 |
| SW948 | COLON | 32.8 | 30.7 | 1.1 |
| A673 + | EWINGS SARCOMA* | 79.4 | 52 | 1.5 |
| H446 ++ | LUNG | 59.9 | 23.3 | 2.6 |
| NCI H226 + | LUNG | 12 | 6 | 2.0 |
| H460 + | LUNG | 86.1 | 58 | 1.5 |
| A549 + | LUNG | 7.1 | 4.8 | 1.5 |
| H292 | LUNG | 139 | 107 | 1.3 |
| NCI H1155 | LUNG | 7.9 | 6.2 | 1.3 |
| SK-MES-1 | LUNG | 174 | 156 | 1.1 |
| U937 | MONOCYTE | 8.2 | 6.7 | 1.2 |
| | CANCER* | | | |
| MINO + | MANTLE | 15 | 7.6 | 2.0 |
| JEKO | MANTLE | 5.2 | 3.9 | 1.3 |
| COLO 829 ++ | MELANOMA | 16 | 6.9 | 2.3 |
| NCI H929 | MELANOMA | 52.6 | 43.8 | 1.2 |
| EW36 | NHL* | 6.4 | 5.6 | 1.1 |
| OVCAR4 + | OVARIAN | 128 | 61 | 2.1 |
| HELA | OVARIAN | 6.8 | 6.1 | 1.1 |
| SK-OV-3 | OVARIAN | 6.1 | 5.6 | 1.1 |
| CAPAN-1 + | PANCREAS | 130 | 86.9 | 1.5 |
| CAPAN-2 | PANCREAS | 5.9 | 4.7 | 1.3 |
| DU-145 | PROSTRATE | 4 | 3.2 | 1.3 |
| PC3 | PROSTRATE | 3.3 | 2.8 | 1.2 |
| HT144 + | SKIN | 203 | 126 | 1.6 |
| SK-MEL-2 | SKIN | 7.7 | 7 | 1.1 |
| JURKAT | T-CELL ALL* | 6.5 | 5.7 | 1.1 |
| MOLT4 | T-CELL ALL* | 4.8 | 5.1 | 0.9 |

| | |
|---|---|
| Key: | Staining at ++ |
| | Staining at + |
| | No Staining |

### REFERENCES

1. Lu, W., Yamamoto, V., Ortega, B. & Baltimore, D. Mammalian Ryk is a Wnt coreceptor required for stimulation of neurite outgrowth. Cell 119, 97-108 (2004).
2. Keeble, T. R., et al. The Wnt receptor Ryk is required for Wnt5a-mediated axon guidance on the contralateral side of the corpus callosum. J Neurosci 26, 5840-5848 (2006).
3. Chakravadhanula, M., et al. Wnt pathway in atypical teratoid rhabdoid tumors. Neuro Oncol 17, 526-535 (2015).
4. Anastas, J. N. Functional Crosstalk Between WNT Signaling and Tyrosine Kinase Signaling in Cancer. Semin Oncol 42, 820-831 (2015).
5. Roy, J. P., Halford, M. M. & Stacker, S. A. The biochemistry, signalling and disease relevance of RYK and other WNT-binding receptor tyrosine kinases. Growth Factors 36, 15-40 (2018).
6. Mikels, A. J. & Nusse, R. Purified Wnt5a protein activates or inhibits beta-catenin-TCF signaling depending on receptor context. PLOS Biol 4, e115 (2006).
7. Wu, C. H. & Nusse, R. Ligand receptor interactions in the Wnt signaling pathway in Drosophila. J Biol Chem 277, 41762-41769 (2002).
8. Lyu, J., Yamamoto, V. & Lu, W. Cleavage of the Wnt receptor Ryk regulates neuronal differentiation during cortical neurogenesis. Dev Cell 15, 773-780 (2008).
9. Halford, M. M., et al. Ryk-deficient mice exhibit craniofacial defects associated with perturbed Eph receptor crosstalk. Nat Genet 25, 414-418 (2000).
10. Blakely, B. D., et al. Ryk, a receptor regulating Wnt5a-mediated neurogenesis and axon morphogenesis of ventral midbrain dopaminergic neurons. Stem Cells Dev 22, 2132-2144 (2013).
11. Kugathasan, K., et al. Deficiency of the Wnt receptor Ryk causes multiple cardiac and outflow tract defects. Growth Factors 36, 58-68 (2018).
12. Famili, F., et al. The non-canonical Wnt receptor Ryk regulates hematopoietic stem cell repopulation in part by controlling proliferation and apoptosis. Cell Death Dis 7, e2479 (2016).
13. Adamo, A., et al. RYK promotes the stemness of glioblastoma cells via the WNT/beta-catenin pathway. Oncotarget 8, 13476-13487 (2017).
14. Borcherding, N., et al. Paracrine WNT5A Signaling Inhibits Expansion of Tumor-Initiating Cells. Cancer Res 75, 1972-1982 (2015).
15. Kessenbrock, K., et al. Diverse regulation of mammary epithelial growth and branching morphogenesis through noncanonical Wnt signaling. Proc Natl Acad Sci USA 114, 3121-3126 (2017).
16. Fu, Y., Chen, Y., Huang, J., Cai, Z. & Wang, Y. RYK, a receptor of noncanonical Wnt ligand Wnt5a, is positively correlated with gastric cancer tumorigenesis and potential of liver metastasis. Am J Physiol Gastrointest Liver Physiol 318, G352-G360 (2020).

INFORMAL SEQUENCE LISTING
2-D11 CDR H1
SEQ ID NO: 1
GFSLNDYG
2-D11 CDR H2
SEQ ID NO: 2
IWGDGVT
2-D11 CDR H3
SEQ ID NO: 3
QGSGVWFAH
2-D11 CDR L1
SEQ ID NO: 4
QTIVHSNGNTY
2-D11 CDR L2
SEQ ID NO: 5
KVS
2-D11 CDR L3
SEQ ID NO: 6
FQGSHVPYT
2-D11 FR H1
SEQ ID NO: 7
QVQLKESGPGLVAPSQSLSITCSVS
2-D11 FR H2
SEQ ID NO: 8
VNWVRQPPGKDLEWLGM
2-D11 FR H3
SEQ ID NO: 9
EYNSTLKSRLSISKDNSKSQVFLKMNNLQTEDTARYYCVR
2-D11 FR H4
SEQ ID NO: 10
WGQGTLVSVSS
2-D11 FR L1
SEQ ID NO: 11
DVLVTQTPLSLPVSLGDQASISCRSS
2-D11 FR L2
SEQ ID NO: 12
LEWYLQKPGQSPKLLIY
2-D11 FR L3
SEQ ID NO: 13
NRFSGVPDRFSGSGSGTDFTLKISRVEAEDLGIYYC
2-D11 FR L4
SEQ ID NO: 14
FGGGTKLEIK
2-D11 VH
SEQ ID NO: 15
QVQLKESGPGLVAPSQSLSITCSVSGFSLNDYGVNWVRQPPGKDLEWLGMIWGDGVTEYN
STLKSRLSISKDNSKSQVFLKMNNLQTEDTARYYCVRQGSGVWFAHWGQGTLVSVSS
2-D11 VL
SEQ ID NO: 16
DVLVTQTPLSLPVSLGDQASISCRSSQTIVHSNGNTYLEWYLQKPGQSPKLLIYKVSNRFSG
VPDRFSGSGSGTDFTLKISRVEAEDLGIYYCFQGSHVPYTFGGGTKLEIK
7-H10 CDR H1
SEQ ID NO: 17
GYIFTNYD
7-H10 CDR H2
SEQ ID NO: 18
IFPGDDST
7-H10 CDR H3
SEQ ID NO: 19
YHYYGSSLGWSFDV
7-H10 CDR L1
SEQ ID NO: 20
SRISSIN
7-H10 CDR L2
SEQ ID NO: 21
GTS
7-H10 CDR L3
SEQ ID NO: 22
QQWSSYPYT
7-H10 FR H1
SEQ ID NO: 23
QVQLQQSGAELAKPGTSVKLSCKAS
7-H10 FR H2
SEQ ID NO: 24
INWVRQRPEQGLEWIGW
7-H10 FR H3
SEQ ID NO: 25
KYNEKFEGKAALTTDKSSNTAYIQLSRLTSGDSAVYFCTR
7-H10 FR H4
SEQ ID NO: 26
WGAGTSVTVSS
7-H10 FR L1
SEQ ID NO: 27
EIVLTQSPALMAASPGEKVTITCSVS
7-H10 FR L2
SEQ ID NO: 28
LHWYQQKSETSPKTWIY
7-H10 FR L3
SEQ ID NO: 29
NLASGVPSRFSGSGSGTSYSLTISNMEAEDAATYYC
7-H10 FR L4
SEQ ID NO: 30
FGGGTKVEIK
7-H10 VH
SEQ ID NO: 31
QVQLQQSGAELAKPGTSVKLSCKASGYIFTNYDINWVRQRPEQGLEWIGWIFPGDDSTKYN
EKFEGKAALTTDKSSNTAYIQLSRLTSGDSAVYFCTRYHYYGSSLGWSFDVWGAGTSVTVS
S
7-H10 VL
SEQ ID NO: 32
EIVLTQSPALMAASPGEKVTITCSVSSRISSINLHWYQQKSETSPKTWIYGTSNLASGVPSRFS
GSGSGTSYSLTISNMEAEDAATYYCQQWSSYPYTFGGGTKVEIK
11-E9 CDR H1
SEQ ID NO: 33
GFSLNGYG
11-E9 CDR H2
SEQ ID NO: 34
IWGDGIT
11-E9 CDR H3
SEQ ID NO: 35
QGSGVWFAY
11-E9 CDR L1
SEQ ID NO: 36
QTIVHSNGNTY
11-E9 CDR L2
SEQ ID NO: 37
KVS
11-E9 CDR L3
SEQ ID NO: 38
FQGSHVPYT
11-E9 FR H1
SEQ ID NO: 39
QVQLKESGPGLVAPSQSLSITCTVS
11-E9 FR H2
SEQ ID NO: 40
VNWVRQPPGKDLEWLGM
11-E9 FR H3
SEQ ID NO: 41
EFNSALKSRLSISKDNSKSQVFLKMNSLQTEDTARYYCVR
11-E9 FR H4
SEQ ID NO: 42
WGQGTLS
11-E9 FR L1
SEQ ID NO: 43
DVLVTQTPLSLPVSLGDQASISCRSS
11-E9 FR L2
SEQ ID NO: 44
LEWYLQKPGQSPKLLIY
11-E9 FR L3
SEQ ID NO: 45
NRFCGVPDRFSGSGSGTDFTLKISRVEAEDLGVYYC
11-E9 FR L4
SEQ ID NO: 46
FGGGTKLEIK
11-E9 VH
SEQ ID NO: 47
QVQLKESGPGLVAPSQSLSITCTVSGFSLNGYGVNWVRQPPGKDLEWLGMIWGDGITEFNS
ALKSRLSISKDNSKSQVFLKMNSLQTEDTARYYCVRQGSGVWFAYWGQGTLS
11-E9 VL
SEQ ID NO: 48
DVLVTQTPLSLPVSLGDQASISCRSSQTIVHSNGNTYLEWYLQKPGQSPKLLIYKVSNRFCG
VPDRFSGSGSGTDFTLKISRVEAEDLGVYYCFQGSHVPYTFGGGTKLEIK
3-C12 CDR H1
SEQ ID NO: 49
GFSLNGYG
3-C12 CDR H2
SEQ ID NO: 50
IWGDGIT
3-C12 CDR H3
SEQ ID NO: 51
QGSGVWFAY
3-C12 CDR L1
SEQ ID NO: 52
QTIVHSNGNTY
3-C12 CDR L2
SEQ ID NO: 53
KVS
3-C12 CDR L3
SEQ ID NO: 54
FQGSHVPYT
3-C12 FR H1
SEQ ID NO: 55
QVQLKESGPGLVAPSQSLSITCTVS
3-C12 FR H2
SEQ ID NO: 56
VNWVRQPPGKDLEWLGM
3-C12 FR H3
SEQ ID NO: 57
EFNSALKSRLSISKDNSKSQVFLKMNSLQTEDTARYYCVR
3-C12 FR H4
SEQ ID NO: 58
WGQGTLVS
3-C12 FR L1
SEQ ID NO: 59
DVLVTQTPLSLPVSLGDQASISCRSS
3-C12 FR L2
SEQ ID NO: 60
LEWYLQKPGQSPKLLIY
3-C12 FR L3
SEQ ID NO: 61
NRFSGVPDRFSGSGSGTDFTLKISRVEAEDLGVYYC
3-C12 FR L4
SEQ ID NO: 62
FGGGTKLEIK
3-C12 VH
SEQ ID NO: 63
QVQLKESGPGLVAPSQSLSITCTVSGFSLNGYGVNWVRQPPGKDLEWLGMIWGDGITEFNS
ALKSRLSISKDNSKSQVFLKMNSLQTEDTARYYCVRQGSGVWFAYWGQGTLVS
3-C12 VL
SEQ ID NO: 64
DVLVTQTPLSLPVSLGDQASISCRSSQTIVHSNGNTYLEWYLQKPGQSPKLLIYKVSNRFSG
VPDRFSGSGSGTDFTLKISRVEAEDLGVYYCFQGSHVPYTFGGGTKLEIK
2-D11 CDR H1 nucleotide
SEQ ID NO: 65
GGGTTCTCATTAAACGACTATGGT
2-D11 CDR H2 nucleotide
SEQ ID NO: 66
ATTTGGGGTGATGGAGTCACA
2-D11 CDR H3 nucleotide
SEQ ID NO: 67
GTCAGACAGGGGTCTGGTGTCTGGTTTGCTCAC
2-D11 CDR L1 nucleotide
SEQ ID NO: 68
CAGACCATTGTACATAGTAATGGAAACACGTAT
2-D11 CDR L2 nucleotide
SEQ ID NO: 69
AAAGTTTCC
2-D11 CDR L3 nucleotide
SEQ ID NO: 70
TTTCAAGGTTCACATGTTCCGTACACG
2-D11 FR H1 nucleotide
SEQ ID NO: 71
CAGGTGCAGCTGAAGGAGTCAGGACCTGGCCTGGTGGCGCCCTCACAGAGCCTGTCCA
TCACATGTTCCGTCTCA
2-D11 FR H2 nucleotide
SEQ ID NO: 72
GTAAATTGGGTTCGCCAGCCTCCAGGAAAGGATCTGGAGTGGCTGGGAATG
2-D11 FR H3 nucleotide
SEQ ID NO: 73
GAGTATAATTCAACTCTCAAATCCAGACTGAGCATCAGCAAGGACAACTCCAAGAGCC
AAGTTTTCTTAAAAATGAACAATCTGCAAACTGAAGACACAGCCAGGTACTACTGT
2-D11 FR H4 nucleotide
SEQ ID NO: 74
TGGGGCCAAGGGACTCTGGTCAGTGTCTCTTCA
2-D11 FR L1 nucleotide
SEQ ID NO: 75
GATGTTTTGGTGACCCAAACTCCACTCTCCCTGCCTGTCAGTCTTGGAGATCAAGCCTCC
ATCTCTTGCAGATCTAGT
2-D11 FR L2 nucleotide
SEQ ID NO: 76
TTAGAATGGTACCTGCAGAAACCAGGCCAGTCTCCAAAGCTCCTAATCTAC
2-D11 FR L3 nucleotide
SEQ ID NO: 77
AACCGATTTTCTGGGGTCCCAGACAGGTTCAGTGGCAGTGGATCAGGCACAGATTTCAC
ACTCAAGATCAGCAGAGTGGAGGCTGAGGATCTGGGAATTTATTACTGC
2-D11 FR L4 nucleotide
SEQ ID NO: 78
TTCGGAGGGGGGACCAAGCTGGAAATAAAA
2-D11 VH nucleotide
SEQ ID NO: 79
CAGGTGCAGCTGAAGGAGTCAGGACCTGGCCTGGTGGCGCCCTCACAGAGCCTGTCCA
TCACATGTTCCGTCTCAGGGTTCTCATTAAACGACTATGGTGTAAATTGGGTTCGCCAG
CCTCCAGGAAAGGATCTGGAGTGGCTGGGAATGATTTGGGGTGATGGAGTCACAGAGT
ATAATTCAACTCTCAAATCCAGACTGAGCATCAGCAAGGACAACTCCAAGAGCCAAGT
TTTCTTAAAAATGAACAATCTGCAAACTGAAGACACAGCCAGGTACTACTGTGTCAGAC
AGGGGTCTGGTGTCTGGTTTGCTCACTGGGGCCAAGGGACTCTGGTCAGTGTCTCTTCA
2-D11 VL nucleotide
SEQ ID NO: 80
GATGTTTTGGTGACCCAAACTCCACTCTCCCTGCCTGTCAGTCTTGGAGATCAAGCCTCC
ATCTCTTGCAGATCTAGTCAGACCATTGTACATAGTAATGGAAACACGTATTTAGAATG
GTACCTGCAGAAACCAGGCCAGTCTCCAAAGCTCCTAATCTACAAAGTTTCCAACCGAT
TTTCTGGGGTCCCAGACAGGTTCAGTGGCAGTGGATCAGGCACAGATTTCACACTCAAG
ATCAGCAGAGTGGAGGCTGAGGATCTGGGAATTTATTACTGCTTTCAAGGTTCACATGT
TCCGTACACGTTCGGAGGGGGGACCAAGCTGGAAATAAAA
7-H10 CDR H1 nucleotide
SEQ ID NO: 81
GGCTACATCTTCACAAACTATGAT
7-H10 CDR H2 nucleotide
SEQ ID NO: 82
ATTTTTCCTGGAGATGATAGTACT
7-H10 CDR H3 nucleotide
SEQ ID NO: 83
ACAAGATATCATTACTACGGTAGTTCCTTGGGGTGGTCCTTCGATGTC
7-H10 CDR L1 nucleotide
SEQ ID NO: 84
TCAAGAATAAGTTCCATTAAC
7-H10 CDR L2 nucleotide
SEQ ID NO: 85
GGCACATCC
7-H10 CDR L3 nucleotide
SEQ ID NO: 86
CAACAGTGGAGTAGTTATCCGTACACG
7-H10 FR H1 nucleotide
SEQ ID NO: 87
CAGGTTCAGCTGCAGCAGTCTGGAGCTGAACTGGCAAAGCCTGGGACTTCAGTGAAAT
TGTCCTGCAAGGCTTCT
7-H10 FR H2 nucleotide
SEQ ID NO: 88
ATAAACTGGGTGAGGCAGAGGCCTGAACAGGGACTTGAGTGGATTGGATGG
7-H10 FR H3 nucleotide
SEQ ID NO: 89
AAGTACAATGAGAAATTCGAGGGCAAGGCCGCACTGACTACAGACAAGTCCTCCAACA
CAGCCTACATACAACTCAGCAGACTGACATCTGGGGACTCAGCTGTCTATTTCTGT
7-H10 FR H4 nucleotide
SEQ ID NO: 90
TGGGGCGCAGGGACCTCGGTCACCGTCTCCTCA
7-H10 FR L1 nucleotide
SEQ ID NO: 91
GAAATTGTGCTCACCCAGTCTCCAGCACTCATGGCTGCATCTCCAGGGGAGAAGGTCAC
CATCACCTGCAGTGTCAGT
7-H10 FR L2 nucleotide
SEQ ID NO: 92
TTGCACTGGTACCAGCAGAAGTCAGAAACCTCCCCCAAAACCTGGATTTAT
7-H10 FR L3 nucleotide
SEQ ID NO: 93
AACCTGGCTTCTGGAGTCCCTAGTCGCTTCAGTGGCAGTGGATCTGGGACCTCTTATTCT
CTCACAATCAGCAACATGGAGGCTGAAGATGCTGCCACTTATTACTGT
7-H10 FR L4 nucleotide
SEQ ID NO: 94
TTCGGAGGGGGGACCAAGGTGGAAATAAAA
7-H10 VH nucleotide
SEQ ID NO: 95
CAGGTTCAGCTGCAGCAGTCTGGAGCTGAACTGGCAAAGCCTGGGACTTCAGTGAAAT
TGTCCTGCAAGGCTTCTGGCTACATCTTCACAAACTATGATATAAACTGGGTGAGGCAG
AGGCCTGAACAGGGACTTGAGTGGATTGGATGGATTTTTCCTGGAGATGATAGTACTAA
GTACAATGAGAAATTCGAGGGCAAGGCCGCACTGACTACAGACAAGTCCTCCAACACA
GCCTACATACAACTCAGCAGACTGACATCTGGGGACTCAGCTGTCTATTTCTGTACAAG
ATATCATTACTACGGTAGTTCCTTGGGGTGGTCCTTCGATGTCTGGGGCGCAGGGACCT
CGGTCACCGTCTCCTCA
7-H10 VL nucleotide
SEQ ID NO: 96
GAAATTGTGCTCACCCAGTCTCCAGCACTCATGGCTGCATCTCCAGGGGAGAAGGTCAC
CATCACCTGCAGTGTCAGTTCAAGAATAAGTTCCATTAACTTGCACTGGTACCAGCAGA
AGTCAGAAACCTCCCCCAAAACCTGGATTTATGGCACATCCAACCTGGCTTCTGGAGTC
CCTAGTCGCTTCAGTGGCAGTGGATCTGGGACCTCTTATTCTCTCACAATCAGCAACAT
GGAGGCTGAAGATGCTGCCACTTATTACTGTCAACAGTGGAGTAGTTATCCGTACACGT
TCGGAGGGGGGACCAAGGTGGAAATAAAA
11-E9 CDR H1 nucleotide
SEQ ID NO: 97
GGGTTCTCATTAAACGGCTATGGT
11-E9 CDR H2 nucleotide
SEQ ID NO: 98
ATCTGGGGTGATGGAATCACA
11-E9 CDR H3 nucleotide
SEQ ID NO: 99
GTCAGACAGGGGTCTGGTGTCTGGTTTGCTTAC
11-E9 CDR L1 nucleotide
SEQ ID NO: 100
CAGACCATTGTACATAGTAATGGAAACACCTAT
11-E9 CDR L2 nucleotide
SEQ ID NO: 101
AAAGTTTCC
11-E9 CDR L3 nucleotide
SEQ ID NO: 102
TTTCAAGGTTCACATGTTCCGTACACG
11-E9 FR H1 nucleotide
SEQ ID NO: 103
CAGGTGCAGCTGAAGGAGTCAGGACCTGGCCTGGTGGCGCCCTCACAGAGCCTGTCCA
TCACATGTACCGTCTCA
11-E9 FR H2 nucleotide
SEQ ID NO: 104
GTAAACTGGGTTCGCCAGCCTCCAGGAAAGGATCTGGAGTGGCTGGGAATG
11-E9 FR H3 nucleotide
SEQ ID NO: 105
GAGTTTAATTCAGCTCTCAAATCCAGACTGAGCATCAGCAAGGACAACTCCAAGAGCC
AAGTTTTCTTAAAAATGAACAGTCTGCAAACTGAAGACACAGCCAGGTACTACTGT
11-E9 FR H4 nucleotide
SEQ ID NO: 106
TGGGGCCAAGGGACTCTGTCA
11-E9 FR L1 nucleotide
SEQ ID NO: 107
GATGTTTTGGTGACCCAAACTCCACTCTCCCTGCCTGTCAGTCTTGGAGATCAAGCCTCC
ATCTCTTGCAGATCTAGT
11-E9 FR L2 nucleotide
SEQ ID NO: 108
TTAGAATGGTACCTGCAGAAACCAGGCCAGTCTCCAAAGCTCTTGATTTAC
11-E9 FR L3 nucleotide
SEQ ID NO: 109
AACCGATTTTGTGGGGTCCCAGACAGGTTCAGTGGCAGTGGATCAGGCACAGATTTCAC
ACTCAAGATCAGCAGAGTGGAGGCTGAGGATCTGGGAGTTTATTACTGC
11-E9 FR L4 nucleotide
SEQ ID NO: 110
TTCGGAGGGGGGACCAAGCTGGAAATAAAA
11-E9 VH nucleotide
SEQ ID NO: 111
CAGGTGCAGCTGAAGGAGTCAGGACCTGGCCTGGTGGCGCCCTCACAGAGCCTGTCCA
TCACATGTACCGTCTCAGGGTTCTCATTAAACGGCTATGGTGTAAACTGGGTTCGCCAG
CCTCCAGGAAAGGATCTGGAGTGGCTGGGAATGATCTGGGGTGATGGAATCACAGAGT
TTAATTCAGCTCTCAAATCCAGACTGAGCATCAGCAAGGACAACTCCAAGAGCCAAGT
TTTCTTAAAAATGAACAGTCTGCAAACTGAAGACACAGCCAGGTACTACTGTGTCAGAC
AGGGGTCTGGTGTCTGGTTTGCTTACTGGGGCCAAGGGACTCTGTCA
11-E9 VL nucleotide
SEQ ID NO: 112
GATGTTTTGGTGACCCAAACTCCACTCTCCCTGCCTGTCAGTCTTGGAGATCAAGCCTCC
ATCTCTTGCAGATCTAGTCAGACCATTGTACATAGTAATGGAAACACCTATTTAGAATG
GTACCTGCAGAAACCAGGCCAGTCTCCAAAGCTCTTGATTTACAAAGTTTCCAACCGAT
TTTGTGGGGTCCCAGACAGGTTCAGTGGCAGTGGATCAGGCACAGATTTCACACTCAAG
ATCAGCAGAGTGGAGGCTGAGGATCTGGGAGTTTATTACTGCTTTCAAGGTTCACATGT
TCCGTACACGTTCGGAGGGGGGACCAAGCTGGAAATAAAA
3-C12 CDR H1 nucleotide
SEQ ID NO: 113
GGGTTCTCATTAAACGGCTATGGT
3-C12 CDR H2 nucleotide
SEQ ID NO: 114
ATCTGGGGTGATGGAATCACA
3-C12 CDR H3 nucleotide
SEQ ID NO: 115
CAGGGGTCTGGTGTCTGGTTTGCTTAC
3-C12 CDR L1 nucleotide
SEQ ID NO: 116
CAGACCATTGTACATAGTAATGGAAACACCTAT
3-C12 CDR L2 nucleotide
SEQ ID NO: 117
AAAGTTTCC
3-C12 CDR L3 nucleotide
SEQ ID NO: 118
TTTCAAGGTTCACATGTTCCGTACACG
3-C12 FR H1 nucleotide
SEQ ID NO: 119
CAGGTGCAGCTGAAGGAGTCAGGACCTGGCCTGGTGGCGCCCTCACAGAGCCTGTCCA
TCACATGTACCGTCTCA
3-C12 FR H2 nucleotide
SEQ ID NO: 120
GTAAACTGGGTTCGCCAGCCTCCAGGAAAGGATCTGGAGTGGCTGGGAATG
3-C12 FR H3 nucleotide
SEQ ID NO: 121
GAGTTTAATTCAGCTCTCAAATCCAGACTGAGCATCAGCAAGGACAACTCCAAGAGCC
AAGTTTTCTTAAAAATGAACAGTCTGCAAACTGAAGACACAGCCAGGTACTACTGTGTC
AGA
3-C12 FR H4 nucleotide
SEQ ID NO: 122
TGGGGCCAAGGGACTCTGGTCAGT
3-C12 FR L1 nucleotide
SEQ ID NO: 123
GATGTTTTGGTGACCCAAACTCCACTCTCCCTGCCTGTCAGTCTTGGAGATCAAGCCTCC
ATCTCTTGCAGATCTAGT
3-C12 FR L2 nucleotide
SEQ ID NO: 124
TTAGAATGGTACCTGCAGAAACCAGGCCAGTCTCCAAAGCTCCTGATTTAC
3-C12 FR L3 nucleotide
SEQ ID NO: 125
AACCGATTTTCTGGGGTCCCAGACAGGTTCAGTGGCAGTGGATCAGGCACAGATTTCAC
ACTCAAGATCAGCAGAGTGGAGGCTGAGGATCTGGGAGTTTATTACTGC
3-C12 FR L4 nucleotide
SEQ ID NO: 126
TTCGGAGGGGGGACCAAGCTGGAAATAAAA
3-C12 VH nucleotide
SEQ ID NO: 127
CAGGTGCAGCTGAAGGAGTCAGGACCTGGCCTGGTGGCGCCCTCACAGAGCCTGTCCA
TCACATGTACCGTCTCAGGGTTCTCATTAAACGGCTATGGTGTAAACTGGGTTCGCCAG
CCTCCAGGAAAGGATCTGGAGTGGCTGGGAATGATCTGGGGTGATGGAATCACAGAGT
TTAATTCAGCTCTCAAATCCAGACTGAGCATCAGCAAGGACAACTCCAAGAGCCAAGT
TTTCTTAAAAATGAACAGTCTGCAAACTGAAGACACAGCCAGGTACTACTGTGTCAGAC
AGGGGTCTGGTGTCTGGTTTGCTTACTGGGGCCAAGGGACTCTGGTCAGT
3-C12 VL nucleotide
SEQ ID NO: 128
GATGTTTTGGTGACCCAAACTCCACTCTCCCTGCCTGTCAGTCTTGGAGATCAAGCCTCC
ATCTCTTGCAGATCTAGTCAGACCATTGTACATAGTAATGGAAACACCTATTTAGAATG
GTACCTGCAGAAACCAGGCCAGTCTCCAAAGCTCCTGATTTACAAAGTTTCCAACCGAT
TTTCTGGGGTCCCAGACAGGTTCAGTGGCAGTGGATCAGGCACAGATTTCACACTCAAG
ATCAGCAGAGTGGAGGCTGAGGATCTGGGAGTTTATTACTGCTTTCAAGGTTCACATGT
TCCGTACACGTTCGGAGGGGGGACCAAGCTGGAAATAAAA
Amino Acids 1-181 of human RYK extracellular domain
SEQ ID NO: 129
MRGAARLGRPGRSCLPGARGLRAPPPPPLLLLLALLPLLPAPGAAAAPAPRPPELQSASAGP
SVSLYLSEDEVRRLIGLDAELYYVRNDLISHYALSFSLLVPSETNFLHFTWHAKSKVEYKLG
FQVDNVLAMDMPQVNISVQGEVPRTLSVFRVELSCTGKVDSEVMILMQLNLTVNSSK
Amino Acids 48-57 of human RYK extracellular domain
SEQ ID NO: 130
PAPRPPELQS
Mouse RYK extracellular domain
SEQ ID NO: 131
MRAGRGGVPGSGGLRAPPPPLLLLLLAMLPAAAPRSPALAAAPAGPSVSLYLSEDEVRRLL
GLDAELYYVRNDLISHYALSFNLLVPSETNFLHFTWHAKSKVEYKLGFQVDNFVAMGMPQ
VNISAQGEVPRTLSVFRVELSCTGKVDSEVMILMQLNLTVNSSKNFTVLNFKRRKMCYKKL
EEVKTSALDKNTSRTIYDPVHAAPTTSTRVFY
Human RYK extracellular domain
SEQ ID NO: 132
MRGAARLGRPGRSCLPGARGLRAPPPPPLLLLLALLPLLPAPGAAAAPAPRPPELQSASAGP
SVSLYLSEDEVRRLIGLDAELYYVRNDLISHYALSFSLLVPSETNFLHFTWHAKSKVEYKLG
FQVDNVLAMDMPQVNISVQGEVPRTLSVFRVELSCTGKVDSEVMILMQLNLTVNSSKNFT
VLNFKRRKMCYKKLEEVKTSALDKNTSRTIYDPVHAAPTTSTRVFY
Mouse IGHV2-6
SEQ ID NO: 133
QVQLKESGPGLVAPSQSLSITCTVSGFSLTGYGVNWVRQPPGKGLEWLGMIWGDGSTDYN
SALKSRLSISKDNSKSQVFLKMNSLQTDDTARYYCAR
Mouse IGKV1-117
SEQ ID NO: 134
DVLMTQTPLSLPVSLGDQASISCRSSQSIVHSNGNTYLEWYLQKPGQSPKLLIYKVSNRFSG
VPDRFSGSGSGTDFTLKISRVEAEDLGVYYCFQGSHVP
Mouse IGHV1-85
SEQ ID NO: 135
QVQLQQSGPELVKPGASVKLSCKASGYTFTSYDINWVKQRPGQGLEWIGWIYPRDGSTKY
NEKFKGKATLTVDTSSSTAYMELHSLTSEDSAVYFCAR
Mouse IGKV4-53
SEQ ID NO: 136
EIVLTQSPALMAASPGEKVTITCSVSSSISSSNLHWYQQKSETSPKPWIYGTSNLASGVPVRF
SGSGSGTSYSLTISSMEAEDAATYYCQQWSSYPL

### P Embodiments

P Embodiment 1. An anti-related-to-receptor tyrosine kinase (Ryk) antibody comprising a heavy chain variable domain and a light chain variable domain, wherein said heavy chain variable domain comprises: a CDR H1 as set forth in SEQ ID NO:1, a CDR H2 as set forth in SEQ ID NO:2 and a CDR H3 as set forth in SEQ ID NO:3; and wherein said light chain variable domain comprises: a CDR L1 as set forth in SEQ ID NO:4, a CDR L2 as set forth in SEQ ID NO:5, and a CDR L3 as set forth in SEQ ID NO:6.

P Embodiment 2. The antibody of P embodiment 1, wherein said heavy chain variable domain comprises the sequence of SEQ ID NO:21.

P Embodiment 3. The antibody of P embodiment 1 or 2, wherein said light chain variable domain comprises the sequence of SEQ ID NO:22.

P Embodiment 4. An anti-related-to-receptor tyrosine kinase (Ryk) antibody comprising a heavy chain variable domain and a light chain variable domain, wherein said heavy chain variable domain comprises: a CDR H1 as set forth in SEQ ID NO:7, a CDR H2 as set forth in SEQ ID NO:8 and a CDR H3 as set forth in SEQ ID NO:9; and wherein said light chain variable domain comprises: a CDR L1 as set forth in SEQ ID NO: 10, a CDR L2 as set forth in SEQ ID NO:11, and a CDR L3 as set forth in SEQ ID NO:12.

P Embodiment 5. The antibody of P embodiment 4, wherein said heavy chain variable domain comprises the sequence of SEQ ID NO:31.

P Embodiment 6. The antibody of P embodiment 4 or 5, wherein said heavy chain variable domain comprises the sequence of SEQ ID NO:32.

P Embodiment 7. A method of treating cancer in a subject in need thereof, said method comprising administering to a subject a therapeutically effective amount of an antibody of any one of P embodiments 1 to 6.

### Embodiments

Embodiment 1. An anti-RYK antibody comprising a light chain variable domain and a heavy chain variable domain, wherein said heavy chain variable domain comprises a CDR H1 as set forth in SEQ ID NO: 1, a CDR H2 as set forth in SEQ ID NO:2, and a CDR H3 as set forth in SEQ ID NO: 3; and wherein said light chain variable domain comprises a CDR L1 as set forth in SEQ ID NO: 4, a CDR L2 as set forth in SEQ ID NO:5 and a CDR L3 as set forth in SEQ ID NO:6.

Embodiment 2. The anti-RYK antibody of embodiment 1 wherein said heavy chain variable domain comprises the sequence of SEQ ID NO: 15.

Embodiment 3. The anti-RYK antibody of any one of embodiments 1-2, wherein said light chain variable domain comprises the sequence of SEQ ID NO: 16.

Embodiment 4. The anti-RYK antibody of any one of embodiments 1-3, wherein said anti-RYK antibody has a K_{D} of about 2 pM to about 2 nM.

Embodiment 5. The anti-RYK antibody of any one of embodiments 1-4, wherein said anti-RYK antibody has a K_{D} of about 513 pM.

Embodiment 6. An anti-RYK antibody comprising a light chain variable domain and a heavy chain variable domain, wherein said heavy chain variable domain comprises a CDR H1 as set forth in SEQ ID NO: 17, a CDR H2 as set forth in SEQ ID NO: 18, and a CDR H3 as set forth in SEQ ID NO:19; and wherein said light chain variable domain comprises a CDR L1 as set forth in SEQ ID NO:20, a CDR L2 as set forth in SEQ ID NO:21 and a CDR L3 as set forth in SEQ ID NO: 22.

Embodiment 7. The anti-RYK antibody of embodiment 6, wherein said heavy chain variable domain comprises the sequence of SEQ ID NO:31.

Embodiment 8. The anti-RYK antibody of any one of embodiments 6-7, wherein said light chain variable domain comprises the sequence of SEQ ID NO:32.

Embodiment 9. The anti-RYK antibody of any one of embodiments 6-8, wherein said anti-RYK antibody has a K_{D} of about 6 nM to about 17 nM.

Embodiment 10. The anti-RYK antibody of any one of embodiments 6-9, wherein said anti-RYK antibody has a K_{D} of about 10 nM.

Embodiment 11. An anti-RYK antibody comprising a light chain variable domain and a heavy chain variable domain, wherein said heavy chain variable domain comprises a CDR H1 as set forth in SEQ ID NO:33, a CDR H2 as set forth in SEQ ID NO:34, and a CDR H3 as set forth in SEQ ID NO:35; and wherein said light chain variable domain comprises a CDR L1 as set forth in SEQ ID NO:36, a CDR L2 as set forth in SEQ ID NO:37 and a CDR L3 as set forth in SEQ ID NO: 38.

Embodiment 12. The anti-RYK antibody of embodiment 11, wherein said heavy chain variable domain comprises the sequence of SEQ ID NO:47.

Embodiment 13. The anti-RYK antibody of any one of embodiments 11-12, wherein said light chain variable domain comprises the sequence of SEQ ID NO:48.

Embodiment 14. An anti-RYK antibody comprising a light chain variable domain and a heavy chain variable domain, wherein said heavy chain variable domain comprises a CDR H1 as set forth in SEQ ID NO:49, a CDR H2 as set forth in SEQ ID NO:50, and a CDR H3 as set forth in SEQ ID NO:51; and wherein said light chain variable domain comprises a CDR L1 as set forth in SEQ ID NO:52, a CDR L2 as set forth in SEQ ID NO:53 and a CDR L3 as set forth in SEQ ID NO: 54.

Embodiment 15. The anti-RYK antibody of embodiment 14, wherein said heavy chain variable domain comprises the sequence of SEQ ID NO:63.

Embodiment 16. The anti-RYK antibody of any one of embodiments 14-15, wherein said light chain variable domain comprises the sequence of SEQ ID NO:64.

Embodiment 17. The anti-RYK antibody of any one of embodiments 1-16, wherein said anti-RYK antibody is a chimeric antibody.

Embodiment 18. The anti-RYK antibody of any one of embodiments 1-17, wherein said anti-RYK antibody is a Fab' fragment.

Embodiment 19. The anti-RYK antibody of any one of embodiments 1-18, wherein said anti-RYK antibody is an IgG.

Embodiment 20. The anti-RYK antibody of any one of embodiments 1-17, wherein said light chain variable domain and said heavy chain variable domain form part of a scFv.

Embodiment 21. The anti-RYK antibody of any one of embodiments 1-20, wherein said anti-RYK antibody is capable of binding a RYK protein.

Embodiment 22. The anti-RYK antibody of any one of embodiments 1-21, wherein said anti-RYK antibody binds an extracellular RYK domain.

Embodiment 23. The anti-RYK antibody of any one of embodiments 1-22, wherein said anti-RYK antibody binds an extracellular RYK domain comprising the amino acid sequence of SEQ ID NO: 129.

Embodiment 24. The anti-RYK antibody of any one of embodiments 1-23, wherein said anti-RYK antibody binds an amino acid sequence corresponding to amino acid residues 48 through 57 of SEQ ID NO: 129.

Embodiment 25. The anti-RYK antibody of any one of embodiments 1-21, wherein said anti-RYK antibody is bound to a RYK protein.

Embodiment 26. The anti-RYK antibody of any one of embodiments 21-25, wherein said RYK protein is a human RYK protein.

Embodiment 27. The anti-RYK antibody of any one of embodiments 21-26, wherein said RYK protein comprises the sequence of SEQ ID NO:130.

Embodiment 28. The anti-RYK antibody of any one of embodiments 21-27, wherein said RYK protein does not bind to a mouse RYK protein.

Embodiment 29. The anti-RYK antibody of any one of embodiments 21-28, wherein said anti-RYK antibody does not bind a RYK protein comprising an amino acid sequence corresponding to amino acid residues 32 through 41 of SEQ ID NO: 131.

Embodiment 30. The anti-RYK antibody of any one of embodiments 25-28, wherein said RYK protein forms part of a cell.

Embodiment 31. The anti-RYK antibody of any one of embodiments 21-30, wherein said RYK protein is expressed on the surface of a cell.

Embodiment 32. An anti-RYK antibody, wherein said anti-RYK antibody binds the same epitope as an antibody comprising: a heavy chain variable domain comprising a CDR H1 as set forth in SEQ ID NO:1, a CDR H2 as set forth in SEQ ID NO:2, and a CDR H3 as set forth in SEQ ID NO: 3, and a light chain variable domain comprising a CDR L1 as set forth in SEQ ID NO:4, a CDR L2 as set forth in SEQ ID NO:5 and a CDR L3 as set forth in SEQ ID NO:6.

Embodiment 33. An anti-RYK antibody, wherein said anti-RYK antibody binds the same epitope as an antibody comprising: a heavy chain variable domain comprising a CDR H1 as set forth in SEQ ID NO:17, a CDR H2 as set forth in SEQ ID NO:18, and a CDR H3 as set forth in SEQ ID NO: 19, and a light chain variable domain comprising a CDR L1 as set forth in SEQ ID NO:20, a CDR L2 as set forth in SEQ ID NO:21 and a CDR L3 as set forth in SEQ ID NO:22.

Embodiment 34. An anti-RYK antibody, wherein said anti-RYK antibody binds the same epitope as an antibody comprising: a heavy chain variable domain comprising a CDR H1 as set forth in SEQ ID NO:33, a CDR H2 as set forth in SEQ ID NO:34, and a CDR H3 as set forth in SEQ ID NO: 35, and a light chain variable domain comprising a CDR L1 as set forth in SEQ ID NO:36, a CDR L2 as set forth in SEQ ID NO:37 and a CDR L3 as set forth in SEQ ID NO:38.

Embodiment 35. An anti-RYK antibody, wherein said anti-RYK antibody binds the same epitope as an antibody comprising: a heavy chain variable domain comprising a CDR H1 as set forth in SEQ ID NO:49, a CDR H2 as set forth in SEQ ID NO:50, and a CDR H3 as set forth in SEQ ID NO: 51, and a light chain variable domain comprising a CDR L1 as set forth in SEQ ID NO:52, a CDR L2 as set forth in SEQ ID NO:53 and a CDR L3 as set forth in SEQ ID NO:54.

Embodiment 36. The anti-RYK antibody of any one of embodiments 1-35, wherein said anti-RYK antibody is attached to a therapeutic or a diagnostic moiety.

Embodiment 37. An isolated nucleic acid encoding an anti-RYK antibody of any one of embodiments 1-36.

Embodiment 38. A cell comprising an anti-RYK antibody of any one of embodiments 1-36, or a nucleic acid of embodiment 37.

Embodiment 39. A pharmaceutical composition comprising a therapeutically effective amount of an antibody of any of embodiments 1-36 and a pharmaceutically acceptable excipient.

Embodiment 40. A method of forming an antibody capable of binding to a RYK protein, said method comprising immunizing a mammal with a peptide comprising the sequence of SEQ ID NO: 129.

Embodiment 41. A method of detecting a RYK-expressing cell, said method comprising (i) contacting a RYK-expressing cell with an antibody of any one of embodiments 1-36; (ii) and detecting binding of said antibody to a RYK protein expressed by said cell.

Embodiment 42. The method of embodiment 41, wherein said antibody is attached to a detectable moiety.

Embodiment 43. The method of embodiment 41 or 42, wherein said RYK-expressing cell is in a biological sample.

Embodiment 44. The method of embodiment 41, wherein the biological sample is whole blood, blood fractions or products, tissue, or cultured cells.

Embodiment 45. The method of any one of embodiments 41-44, wherein said RYK-expressing cell is a cancer cell.

Embodiment 46. The method of embodiment 45, wherein said cancer cell is a bladder cancer cell, a brain cancer cell, a breast cancer cell, a chronic myeloid leukemia (CML) cell, a colon cancer cell, a Ewing's sarcoma cell, a lung cancer cell, a mantle cell lymphoma cell, an ovarian cancer cell, a pancreas cancer cell, a skin cancer cell or a melanoma cell.

Embodiment 47. A method of treating cancer in a subject in need thereof, said method comprising administering to a subject a therapeutically effective amount of an anti-RYK antibody of any one of embodiments 1-36.

Embodiment 48. The method of embodiment 47, wherein the cancer is bladder cancer, brain cancer, breast cancer, chronic myeloid leukemia (CML), colon cancer, Ewing's sarcoma, lung cancer, mantle cell lymphoma, ovarian cancer, pancreas cancer, skin cancer, or melanoma.

Embodiment 49. A method of identifying an anti-RYK antibody, the method comprising: (i) contacting an antibody with a first RYK polypeptide comprising an amino acid sequence corresponding to amino acid residues 48 through 57 of SEQ ID NO: 129; (ii) detecting said antibody binding to said first RYK polypeptide; (iii) contacting said antibody with a second RYK polypeptide not comprising an amino acid sequence corresponding to amino acid residues 48 through 57 of SEQ ID NO: 129; and (iv) detecting said antibody not binding to said second RYK polypeptide, thereby identifying an anti-RYK antibody.

Embodiment 50. The method of embodiment 49, wherein said antibody is a chimeric antibody.

Embodiment 51. The method of embodiment 49 or 50, wherein said antibody is a Fab' fragment.

Embodiment 52. The method of embodiment 49 or 50, wherein said antibody is a single chain antibody.

What is claimed is:

1. An anti-RYK antibody comprising a heavy chain variable domain and a light chain variable domain, wherein said heavy chain variable domain comprises a CDR H1 as set forth in SEQ ID NO:1, a CDR H2 as set forth in SEQ ID NO:2, and a CDR H3 as set forth in SEQ ID NO: 3; and
wherein said light chain variable domain comprises a CDR L1 as set forth in SEQ ID NO: 4, a CDR L2 as set forth in SEQ ID NO:5 and a CDR L3 as set forth in SEQ ID NO:6.

2. The anti-RYK antibody of claim 1 wherein said heavy chain variable domain comprises the sequence of SEQ ID NO:15.

3. The anti-RYK antibody of any one of claims 1-2, wherein said light chain variable domain comprises the sequence of SEQ ID NO: 16.

4. The anti-RYK antibody of any one of claims 1-3, wherein said anti-RYK antibody has a K_{D} of about 2 pM to about 2 nM.

5. The anti-RYK antibody of any one of claims 1-4, wherein said anti-RYK antibody has a K_{D} of about 513 pM.

6. An anti-RYK antibody comprising a heavy chain variable domain and a light chain variable domain, wherein said heavy chain variable domain comprises a CDR H1 as set forth in SEQ ID NO:17, a CDR H2 as set forth in SEQ ID NO: 18, and a CDR H3 as set forth in SEQ ID NO: 19; and
wherein said light chain variable domain comprises a CDR L1 as set forth in SEQ ID NO: 20, a CDR L2 as set forth in SEQ ID NO:21 and a CDR L3 as set forth in SEQ ID NO:22.

7. The anti-RYK antibody of claim 6, wherein said heavy chain variable domain comprises the sequence of SEQ ID NO:31.

8. The anti-RYK antibody of any one of claims 6-7, wherein said light chain variable domain comprises the sequence of SEQ ID NO:32.

9. The anti-RYK antibody of any one of claims 6-8, wherein said anti-RYK antibody has a K_{D} of about 6 nM to about 17 nM.

10. The anti-RYK antibody of any one of claims 6-9, wherein said anti-RYK antibody has a K_{D} of about 10 nM.

11. An anti-RYK antibody comprising a heavy chain variable domain and a light chain variable domain, wherein said heavy chain variable domain comprises a CDR H1 as set forth in SEQ ID NO:33, a CDR H2 as set forth in SEQ ID NO:34, and a CDR H3 as set forth in SEQ ID NO:35; and
wherein said light chain variable domain comprises a CDR L1 as set forth in SEQ ID NO: 36, a CDR L2 as set forth in SEQ ID NO:37 and a CDR L3 as set forth in SEQ ID NO:38.

12. The anti-RYK antibody of claim 11, wherein said heavy chain variable domain comprises the sequence of SEQ ID NO:47.

13. The anti-RYK antibody of any one of claims 11-12, wherein said light chain variable domain comprises the sequence of SEQ ID NO:48.

14. An anti-RYK antibody comprising a heavy chain variable domain and a light chain variable domain, wherein said heavy chain variable domain comprises a CDR H1 as set forth in SEQ ID NO:49, a CDR H2 as set forth in SEQ ID NO:50, and a CDR H3 as set forth in SEQ ID NO:51; and
wherein said light chain variable domain comprises a CDR L1 as set forth in SEQ ID NO: 52, a CDR L2 as set forth in SEQ ID NO:53 and a CDR L3 as set forth in SEQ ID NO:54.

15. The anti-RYK antibody of claim 14, wherein said heavy chain variable domain comprises the sequence of SEQ ID NO:63.

16. The anti-RYK antibody of any one of claims 14-15, wherein said light chain variable domain comprises the sequence of SEQ ID NO:64.

17. The anti-RYK antibody of any one of claims 1-16, wherein said anti-RYK antibody is a chimeric antibody.

18. The anti-RYK antibody of any one of claims 1-17, wherein said anti-RYK antibody is a Fab' fragment.

19. The anti-RYK antibody of any one of claims 1-18, wherein said anti-RYK antibody is an IgG.

20. The anti-RYK antibody of any one of claims 1-17, wherein said light chain variable domain and said heavy chain variable domain form part of a scFv.

21. The anti-RYK antibody of any one of claims 1-20, wherein said anti-RYK antibody is capable of binding a RYK protein.

22. The anti-RYK antibody of any one of claims 1-21, wherein said anti-RYK antibody binds an extracellular RYK domain.

23. The anti-RYK antibody of any one of claims 1-22, wherein said anti-RYK antibody binds an extracellular RYK domain comprising the amino acid sequence of SEQ ID NO: 129.

24. The anti-RYK antibody of any one of claims 1-23, wherein said anti-RYK antibody binds an amino acid sequence corresponding to amino acid residues 48 through 57 of SEQ ID NO: 129.

25. The anti-RYK antibody of any one of claims 1-21, wherein said anti-RYK antibody is bound to a RYK protein.

26. The anti-RYK antibody of any one of claims 21-25, wherein said RYK protein is a human RYK protein.

27. The anti-RYK antibody of any one of claims 21-26, wherein said RYK protein comprises the sequence of SEQ ID NO: 130.

28. The anti-RYK antibody of any one of claims 21-27, wherein said RYK protein does not bind to a mouse RYK protein.

29. The anti-RYK antibody of any one of claims 21-28, wherein said anti-RYK antibody does not bind a RYK protein comprising an amino acid sequence corresponding to amino acid residues 32 through 41 of SEQ ID NO:131.

30. The anti-RYK antibody of any one of claims 25-28, wherein said RYK protein forms part of a cell.

31. The anti-RYK antibody of any one of claims 21-30, wherein said RYK protein is expressed on the surface of a cell.

32. An anti-RYK antibody, wherein said anti-RYK antibody binds the same epitope as an antibody comprising: a heavy chain variable domain comprising a CDR H1 as set forth in SEQ ID NO: 1, a CDR H2 as set forth in SEQ ID NO:2, and a CDR H3 as set forth in SEQ ID NO: 3, and a light chain variable domain comprising a CDR L1 as set forth in SEQ ID NO:4, a CDR L2 as set forth in SEQ ID NO:5 and a CDR L3 as set forth in SEQ ID NO:6.

33. An anti-RYK antibody, wherein said anti-RYK antibody binds the same epitope as an antibody comprising: a heavy chain variable domain comprising a CDR H1 as set forth in SEQ ID NO: 17, a CDR H2 as set forth in SEQ ID NO: 18, and a CDR H3 as set forth in SEQ ID NO: 19, and a light chain variable domain comprising a CDR L1 as set forth in SEQ ID NO: 20, a CDR L2 as set forth in SEQ ID NO:21 and a CDR L3 as set forth in SEQ ID NO:22.

34. An anti-RYK antibody, wherein said anti-RYK antibody binds the same epitope as an antibody comprising: a heavy chain variable domain comprising a CDR H1 as set forth in SEQ ID NO:33, a CDR H2 as set forth in SEQ ID NO:34, and a CDR H3 as set forth in SEQ ID NO:35, and a light chain variable domain comprising a CDR L1 as set forth in SEQ ID NO: 36, a CDR L2 as set forth in SEQ ID NO:37 and a CDR L3 as set forth in SEQ ID NO:38.

35. An anti-RYK antibody, wherein said anti-RYK antibody binds the same epitope as an antibody comprising: a heavy chain variable domain comprising a CDR H1 as set forth in SEQ ID NO:49, a CDR H2 as set forth in SEQ ID NO:50, and a CDR H3 as set forth in SEQ ID NO:51, and a light chain variable domain comprising a CDR L1 as set forth in SEQ ID NO: 52, a CDR L2 as set forth in SEQ ID NO:53 and a CDR L3 as set forth in SEQ ID NO:54.

36. The anti-RYK antibody of any one of claims 1-35, wherein said anti-RYK antibody is attached to a therapeutic or a diagnostic moiety.

37. An isolated nucleic acid encoding an anti-RYK antibody of any one of claims 1-36.

38. A cell comprising an anti-RYK antibody of any one of claims 1-36, or a nucleic acid of claim 37.

39. A pharmaceutical composition comprising a therapeutically effective amount of an antibody of any of claims 1-36 and a pharmaceutically acceptable excipient.

40. A method of forming an antibody capable of binding to a RYK protein, said method comprising immunizing a mammal with a peptide comprising the sequence of SEQ ID NO: 129.

41. A method of detecting a RYK-expressing cell, said method comprising (i) contacting a RYK-expressing cell with an antibody of any one of claims 1-36; (ii) and detecting binding of said antibody to a RYK protein expressed by said cell.

42. The method of claim 41, wherein said antibody is attached to a detectable moiety.

43. The method of claim 41 or 42, wherein said RYK-expressing cell is in a biological sample.

44. The method of claim 41, wherein the biological sample is whole blood, blood fractions or products, tissue, or cultured cells.

45. The method of any one of claims 41-44, wherein said RYK-expressing cell is a cancer cell.

46. The method of claim 45, wherein said cancer cell is a bladder cancer cell, a brain cancer cell, a breast cancer cell, a chronic myeloid leukemia (CML) cell, a colon cancer cell, an Ewing's sarcoma cell, a lung cancer cell, a mantle cell lymphoma cell, an ovarian cancer cell, a pancreas cancer cell, a skin cancer cell or a melanoma cell.

47. A method of treating cancer in a subject in need thereof, said method comprising administering to a subject a therapeutically effective amount of an anti-RYK antibody of any one of claims 1-36.

48. The method of claim 47, wherein the cancer is bladder cancer, brain cancer, breast cancer, chronic myeloid leukemia (CML), colon cancer, Ewing's sarcoma, lung cancer, mantle cell lymphoma, ovarian cancer, pancreas cancer, skin cancer, or melanoma.

49. A method of identifying an anti-RYK antibody, the method comprising:
(i) contacting an antibody with a first RYK polypeptide comprising an amino acid sequence corresponding to amino acid residues 48 through 57 of SEQ ID NO: 129;
(ii) detecting said antibody binding to said first RYK polypeptide;
(iii) contacting said antibody with a second RYK polypeptide not comprising an amino acid sequence corresponding to amino acid residues 48 through 57 of SEQ ID NO:129; and
(iv) detecting said antibody not binding to said second RYK polypeptide, thereby identifying an anti-RYK antibody.

50. The method of claim 49, wherein said antibody is a chimeric antibody.

51. The method of claim 49 or 50, wherein said antibody is a Fab' fragment.

52. The method of claim 49 or 50, wherein said antibody is a single chain antibody.


SEQUENCE LISTING (136 sequences)

SEQ ID NO: 1 - protein, 8 aa, synthetic construct
GFSLNDYG

SEQ ID NO: 2 - protein, 7 aa, synthetic construct
IWGDGVT

SEQ ID NO: 3 - protein, 9 aa, synthetic construct
QGSGVWFAH

SEQ ID NO: 4 - protein, 11 aa, synthetic construct
QTIVHSNGNTY

SEQ ID NO: 5 - skipped

SEQ ID NO: 6 - protein, 9 aa, synthetic construct
FQGSHVPYT

SEQ ID NO: 7 - protein, 25 aa, synthetic construct
QVQLKESGPGLVAPSQSLSITCSVS

SEQ ID NO: 8 - protein, 17 aa, synthetic construct
VNWVRQPPGKDLEWLGM

SEQ ID NO: 9 - protein, 40 aa, synthetic construct
EYNSTLKSRLSISKDNSKSQVFLKMNNLQTEDTARYYCVR

SEQ ID NO: 10 - protein, 11 aa, synthetic construct
WGQGTLVSVSS

SEQ ID NO: 11 - protein, 26 aa, synthetic construct
DVLVTQTPLSLPVSLGDQASISCRSS

SEQ ID NO: 12 - protein, 17 aa, synthetic construct
LEWYLQKPGQSPKLLIY

SEQ ID NO: 13 - protein, 36 aa, synthetic construct
NRFSGVPDRFSGSGSGTDFTLKISRVEAEDLGIYYC

SEQ ID NO: 14 - protein, 10 aa, synthetic construct
FGGGTKLEIK

SEQ ID NO: 15 - protein, 117 aa, synthetic construct
QVQLKESGPGLVAPSQSLSITCSVSGFSLNDYGVNWVRQPPGKDLEWLGMIWGDGVTEYNSTLKSRLSISKDNSKSQVFLKMNNLQTEDTARYYCVRQGSGVWFAHWGQGTLVSVSS

SEQ ID NO: 16 - protein, 112 aa, synthetic construct
DVLVTQTPLSLPVSLGDQASISCRSSQTIVHSNGNTYLEWYLQKPGQSPKLLIYKVSNRFSGVPDRFSGSGSGTDFTLKISRVEAEDLGIYYCFQGSHVPYTFGGGTKLEIK

SEQ ID NO: 17 - protein, 8 aa, synthetic construct
GYIFTNYD

SEQ ID NO: 18 - protein, 8 aa, synthetic construct
IFPGDDST

SEQ ID NO: 19 - protein, 14 aa, synthetic construct
YHYYGSSLGWSFDV

SEQ ID NO: 20 - protein, 7 aa, synthetic construct
SRISSIN

SEQ ID NO: 21 - skipped

SEQ ID NO: 22 - protein, 9 aa, synthetic construct
QQWSSYPYT

SEQ ID NO: 23 - protein, 25 aa, synthetic construct
QVQLQQSGAELAKPGTSVKLSCKAS

SEQ ID NO: 24 - protein, 17 aa, synthetic construct
INWVRQRPEQGLEWIGW

SEQ ID NO: 25 - protein, 40 aa, synthetic construct
KYNEKFEGKAALTTDKSSNTAYIQLSRLTSGDSAVYFCTR

SEQ ID NO: 26 - protein, 11 aa, synthetic construct
WGAGTSVTVSS

SEQ ID NO: 27 - protein, 26 aa, synthetic construct
EIVLTQSPALMAASPGEKVTITCSVS

SEQ ID NO: 28 - protein, 17 aa, synthetic construct
LHWYQQKSETSPKTWIY

SEQ ID NO: 29 - protein, 36 aa, synthetic construct
NLASGVPSRFSGSGSGTSYSLTISNMEAEDAATYYC

SEQ ID NO: 30 - protein, 10 aa, synthetic construct
FGGGTKVEIK

SEQ ID NO: 31 - protein, 123 aa, synthetic construct
QVQLQQSGAELAKPGTSVKLSCKASGYIFTNYDINWVRQRPEQGLEWIGWIFPGDDSTKYNEKFEGKAALTTDKSSNTAYIQLSRLTSGDSAVYFCTRYHYYGSSLGWSFDVWGAGTSVTVSS

SEQ ID NO: 32 - protein, 108 aa, synthetic construct
EIVLTQSPALMAASPGEKVTITCSVSSRISSINLHWYQQKSETSPKTWIYGTSNLASGVPSRFSGSGSGTSYSLTISNMEAEDAATYYCQQWSSYPYTFGGGTKVEIK

SEQ ID NO: 33 - protein, 8 aa, synthetic construct
GFSLNGYG

SEQ ID NO: 34 - protein, 7 aa, synthetic construct
IWGDGIT

SEQ ID NO: 35 - protein, 9 aa, synthetic construct
QGSGVWFAY

SEQ ID NO: 36 - protein, 11 aa, synthetic construct
QTIVHSNGNTY

SEQ ID NO: 37 - skipped

SEQ ID NO: 38 - protein, 9 aa, synthetic construct
FQGSHVPYT

SEQ ID NO: 39 - protein, 25 aa, synthetic construct
QVQLKESGPGLVAPSQSLSITCTVS

SEQ ID NO: 40 - protein, 17 aa, synthetic construct
VNWVRQPPGKDLEWLGM

SEQ ID NO: 41 - protein, 40 aa, synthetic construct
EFNSALKSRLSISKDNSKSQVFLKMNSLQTEDTARYYCVR

SEQ ID NO: 42 - protein, 7 aa, synthetic construct
WGQGTLS

SEQ ID NO: 43 - protein, 26 aa, synthetic construct
DVLVTQTPLSLPVSLGDQASISCRSS

SEQ ID NO: 44 - protein, 17 aa, synthetic construct
LEWYLQKPGQSPKLLIY

SEQ ID NO: 45 - protein, 36 aa, synthetic construct
NRFCGVPDRFSGSGSGTDFTLKISRVEAEDLGVYYC

SEQ ID NO: 46 - protein, 10 aa, synthetic construct
FGGGTKLEIK

SEQ ID NO: 47 - protein, 113 aa, synthetic construct
QVQLKESGPGLVAPSQSLSITCTVSGFSLNGYGVNWVRQPPGKDLEWLGMIWGDGITEFNSALKSRLSISKDNSKSQVFLKMNSLQTEDTARYYCVRQGSGVWFAYWGQGTLS

SEQ ID NO: 48 - protein, 112 aa, synthetic construct
DVLVTQTPLSLPVSLGDQASISCRSSQTIVHSNGNTYLEWYLQKPGQSPKLLIYKVSNRFCGVPDRFSGSGSGTDFTLKISRVEAEDLGVYYCFQGSHVPYTFGGGTKLEIK

SEQ ID NO: 49 - protein, 8 aa, synthetic construct
GFSLNGYG

SEQ ID NO: 50 - protein, 7 aa, synthetic construct
IWGDGIT

SEQ ID NO: 51 - protein, 9 aa, synthetic construct
QGSGVWFAY

SEQ ID NO: 52 - protein, 11 aa, synthetic construct
QTIVHSNGNTY

SEQ ID NO: 53 - skipped

SEQ ID NO: 54 - protein, 9 aa, synthetic construct
FQGSHVPYT

SEQ ID NO: 55 - protein, 25 aa, synthetic construct
QVQLKESGPGLVAPSQSLSITCTVS

SEQ ID NO: 56 - protein, 17 aa, synthetic construct
VNWVRQPPGKDLEWLGM

SEQ ID NO: 57 - protein, 40 aa, synthetic construct; identical to SEQ ID NO: 41

SEQ ID NO: 58 - protein, 8 aa, synthetic construct
WGQGTLVS

SEQ ID NO: 59 - protein, 26 aa, synthetic construct
DVLVTQTPLSLPVSLGDQASISCRSS

SEQ ID NO: 60 - protein, 17 aa, synthetic construct
LEWYLQKPGQSPKLLIY

SEQ ID NO: 61 - protein, 36 aa, synthetic construct
NRFSGVPDRFSGSGSGTDFTLKISRVEAEDLGVYYC

SEQ ID NO: 62 - protein, 10 aa, synthetic construct
FGGGTKLEIK

SEQ ID NO: 63 - protein, 114 aa, synthetic construct
QVQLKESGPGLVAPSQSLSITCTVSGFSLNGYGVNWVRQPPGKDLEWLGMIWGDGITEFNSALKSRLSISKDNSKSQVFLKMNSLQTEDTARYYCVRQGSGVWFAYWGQGTLVS

SEQ ID NO: 64 - protein, 112 aa, synthetic construct
DVLVTQTPLSLPVSLGDQASISCRSSQTIVHSNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 65 - DNA, 24 bp, synthetic construct
  source 1..24: mol_type = other DNA
gggttctcattaaacgactatggt

SEQ ID NO: 66 - DNA, 21 bp, synthetic construct
  source 1..21: mol_type = other DNA
atttggggtgatggagtcaca

SEQ ID NO: 67 - DNA, 33 bp, synthetic construct
  source 1..33: mol_type = other DNA
gtcagacaggggtctggtgtctggtttgctcac

SEQ ID NO: 68 - DNA, 33 bp, synthetic construct
  source 1..33: mol_type = other DNA
cagaccattgtacatagtaatggaaacacgtat

SEQ ID NO: 69 - skipped

SEQ ID NO: 70 - DNA, 27 bp, synthetic construct
  source 1..27: mol_type = other DNA
tttcaaggttcacatgttccgtacacg

SEQ ID NO: 71 - DNA, 75 bp, synthetic construct
  source 1..75: mol_type = other DNA
caggtgcagctgaaggagtcaggacctggcctggtggcgccctcacagagcctgtccatcacatgttccgtctca

SEQ ID NO: 72 - DNA, 51 bp, synthetic construct
  source 1..51: mol_type = other DNA
gtaaattgggttcgccagcctccaggaaaggatctggagtggctgggaatg

SEQ ID NO: 73 - DNA, 114 bp, synthetic construct
  source 1..114: mol_type = other DNA
gagtataattcaactctcaaatccagactgagcatcagcaaggacaactccaagagccaagttttcttaaaaatgaacaatctgcaaactgaagacacagccaggtactactgt

SEQ ID NO: 74 - DNA, 33 bp, synthetic construct
  source 1..33: mol_type = other DNA
tggggccaagggactctggtcagtgtctcttca

SEQ ID NO: 75 - DNA, 78 bp, synthetic construct
  source 1..78: mol_type = other DNA
gatgttttggtgacccaaactccactctccctgcctgtcagtcttggagatcaagcctccatctcttgcagatctagt

SEQ ID NO: 76 - DNA, 51 bp, synthetic construct
  source 1..51: mol_type = other DNA
ttagaatggtacctgcagaaaccaggccagtctccaaagctcctaatctac

SEQ ID NO: 77 - DNA, 108 bp, synthetic construct
  source 1..108: mol_type = other DNA
aaccgattttctggggtcccagacaggttcagtggcagtggatcaggcacagatttcacactcaagatcagcagagtggaggctgaggatctgggaatttattactgc

SEQ ID NO: 78 - DNA, 30 bp, synthetic construct
  source 1..30: mol_type = other DNA
ttcggaggggggaccaagctggaaataaaa

SEQ ID NO: 79 - DNA, 351 bp, synthetic construct
  source 1..351: mol_type = other DNA
caggtgcagctgaaggagtcaggacctggcctggtggcgccctcacagagcctgtccatcacatgttccgtctcagggttctcattaaacgactatggtgtaaattgggttcgccagcctccaggaaaggatctggagtggctgggaatgatttggggtgatggagtcacagagtataattcaactctcaaatccagactgagcatcagcaaggacaactccaagagccaagttttcttaaaaatgaacaatctgcaaactgaagacacagccaggtactactgtgtcagacaggggtctggtgtctggtttgctcactggggccaagggactctggtcagtgtctcttca

SEQ ID NO: 80 - DNA, 336 bp, synthetic construct
  source 1..336: mol_type = other DNA
gatgttttggtgacccaaactccactctccctgcctgtcagtcttggagatcaagcctccatctcttgcagatctagtcagaccattgtacatagtaatggaaacacgtatttagaatggtacctgcagaaaccaggccagtctccaaagctcctaatctacaaagtttccaaccgattttctggggtcccagacaggttcagtggcagtggatcaggcacagatttcacactcaagatcagcagagtggaggctgaggatctgggaatttattactgctttcaaggttcacatgttccgtacacgttcggaggggggaccaagctggaaataaaa

SEQ ID NO: 81 - DNA, 24 bp, synthetic construct
  source 1..24: mol_type = other DNA
ggctacatcttcacaaactatgat

SEQ ID NO: 82 - DNA, 24 bp, synthetic construct
  source 1..24: mol_type = other DNA
atttttcctggagatgatagtact

SEQ ID NO: 83 - DNA, 48 bp, synthetic construct
  source 1..48: mol_type = other DNA
acaagatatcattactacggtagttccttggggtggtccttcgatgtc

SEQ ID NO: 84 - DNA, 21 bp, synthetic construct
  source 1..21: mol_type = other DNA
tcaagaataagttccattaac

SEQ ID NO: 85 - skipped

SEQ ID NO: 86 - DNA, 27 bp, synthetic construct
  source 1..27: mol_type = other DNA
caacagtggagtagttatccgtacacg

SEQ ID NO: 87 - DNA, 75 bp, synthetic construct
  source 1..75: mol_type = other DNA
caggttcagctgcagcagtctggagctgaactggcaaagcctgggacttcagtgaaattgtcctgcaaggcttct

SEQ ID NO: 88 - DNA, 51 bp, synthetic construct
  source 1..51: mol_type = other DNA
ataaactgggtgaggcagaggcctgaacagggacttgagtggattggatgg

SEQ ID NO: 89 - DNA, 114 bp, synthetic construct
  source 1..114: mol_type = other DNA
aagtacaatgagaaattcgagggcaaggccgcactgactacagacaagtcctccaacacagcctacatacaactcagcagactgacatctggggactcagctgtctatttctgt

SEQ ID NO: 90 - DNA, 33 bp, synthetic construct
  source 1..33: mol_type = other DNA
tggggcgcagggacctcggtcaccgtctcctca

SEQ ID NO: 91 - DNA, 78 bp, synthetic construct
  source 1..78: mol_type = other DNA
gaaattgtgctcacccagtctccagcactcatggctgcatctccaggggagaaggtcaccatcacctgcagtgtcagt

SEQ ID NO: 92 - DNA, 51 bp, synthetic construct
  source 1..51: mol_type = other DNA
ttgcactggtaccagcagaagtcagaaacctcccccaaaacctggatttat

SEQ ID NO: 93 - DNA, 108 bp, synthetic construct
  source 1..108: mol_type = other DNA
aacctggcttctggagtccctagtcgcttcagtggcagtggatctgggacctcttattctctcacaatcagcaacatggaggctgaagatgctgccacttattactgt

SEQ ID NO: 94 - DNA, 30 bp, synthetic construct
  source 1..30: mol_type = other DNA
ttcggaggggggaccaaggtggaaataaaa

SEQ ID NO: 95 - DNA, 369 bp, synthetic construct
  source 1..369: mol_type = other DNA
caggttcagctgcagcagtctggagctgaactggcaaagcctgggacttcagtgaaattgtcctgcaaggcttctggctacatcttcacaaactatgatataaactgggtgaggcagaggcctgaacagggacttgagtggattggatggatttttcctggagatgatagtactaagtacaatgagaaattcgagggcaaggccgcactgactacagacaagtcctccaacacagcctacatacaactcagcagactgacatctggggactcagctgtctatttctgtacaagatatcattactacggtagttccttggggtggtccttcgatgtctggggcgcagggacctcggtcaccgtctcctca

SEQ ID NO: 96 - DNA, 324 bp, synthetic construct
  source 1..324: mol_type = other DNA
gaaattgtgctcacccagtctccagcactcatggctgcatctccaggggagaaggtcaccatcacctgcagtgtcagttcaagaataagttccattaacttgcactggtaccagcagaagtcagaaacctcccccaaaacctggatttatggcacatccaacctggcttctggagtccctagtcgcttcagtggcagtggatctgggacctcttattctctcacaatcagcaacatggaggctgaagatgctgccacttattactgtcaacagtggagtagttatccgtacacgttcggaggggggaccaaggtggaaataaaa

SEQ ID NO: 97 - DNA, 24 bp, synthetic construct
  source 1..24: mol_type = other DNA
gggttctcattaaacggctatggt

SEQ ID NO: 98 - DNA, 21 bp, synthetic construct
  source 1..21: mol_type = other DNA
atctggggtgatggaatcaca

SEQ ID NO: 99 - DNA, 33 bp, synthetic construct
  source 1..33: mol_type = other DNA
gtcagacaggggtctggtgtctggtttgcttac

SEQ ID NO: 100 - DNA, 33 bp, synthetic construct
  source 1..33: mol_type = other DNA
cagaccattgtacatagtaatggaaacacctat

SEQ ID NO: 101 - skipped

SEQ ID NO: 102 - DNA, 27 bp, synthetic construct
  source 1..27: mol_type = other DNA
tttcaaggttcacatgttccgtacacg

SEQ ID NO: 103 - DNA, 75 bp, synthetic construct
  source 1..75: mol_type = other DNA
caggtgcagctgaaggagtcaggacctggcctggtggcgccctcacagagcctgtccatcacatgtaccgtctca

SEQ ID NO: 104 - DNA, 51 bp, synthetic construct
  source 1..51: mol_type = other DNA
gtaaactgggttcgccagcctccaggaaaggatctggagtggctgggaatg

SEQ ID NO: 105 - DNA, 114 bp, synthetic construct
  source 1..114: mol_type = other DNA
gagtttaattcagctctcaaatccagactgagcatcagcaaggacaactccaagagccaagttttcttaaaaatgaacagtctgcaaactgaagacacagccaggtactactgt

SEQ ID NO: 106 - DNA, 21 bp, synthetic construct
  source 1..21: mol_type = other DNA
tggggccaagggactctgtca

SEQ ID NO: 107 - DNA, 78 bp, synthetic construct; identical to SEQ ID NO: 75
  source 1..78: mol_type = other DNA

SEQ ID NO: 108 - DNA, 51 bp, synthetic construct
  source 1..51: mol_type = other DNA
ttagaatggtacctgcagaaaccaggccagtctccaaagctcttgatttac

SEQ ID NO: 109 - DNA, 108 bp, synthetic construct
  source 1..108: mol_type = other DNA
aaccgattttgtggggtcccagacaggttcagtggcagtggatcaggcacagatttcacactcaagatcagcagagtggaggctgaggatctgggagtttattactgc

SEQ ID NO: 110 - DNA, 30 bp, synthetic construct; identical to SEQ ID NO: 78
  source 1..30: mol_type = other DNA

SEQ ID NO: 111 - DNA, 339 bp, synthetic construct
  source 1..339: mol_type = other DNA
caggtgcagctgaaggagtcaggacctggcctggtggcgccctcacagagcctgtccatcacatgtaccgtctcagggttctcattaaacggctatggtgtaaactgggttcgccagcctccaggaaaggatctggagtggctgggaatgatctggggtgatggaatcacagagtttaattcagctctcaaatccagactgagcatcagcaaggacaactccaagagccaagttttcttaaaaatgaacagtctgcaaactgaagacacagccaggtactactgtgtcagacaggggtctggtgtctggtttgcttactggggccaagggactctgtca

SEQ ID NO: 112 - DNA, 336 bp, synthetic construct
  source 1..336: mol_type = other DNA
gatgttttggtgacccaaactccactctccctgcctgtcagtcttggagatcaagcctccatctcttgcagatctagtcagaccattgtacatagtaatggaaacacctatttagaatggtacctgcagaaaccaggccagtctccaaagctcttgatttacaaagtttccaaccgattttgtggggtcccagacaggttcagtggcagtggatcaggcacagatttcacactcaagatcagcagagtggaggctgaggatctgggagtttattactgctttcaaggttcacatgttccgtacacgttcggaggggggaccaagctggaaataaaa

SEQ ID NO: 113 - DNA, 24 bp, synthetic construct
  source 1..24: mol_type = other DNA
gggttctcattaaacggctatggt

SEQ ID NO: 114 - DNA, 21 bp, synthetic construct
  source 1..21: mol_type = other DNA
atctggggtgatggaatcaca

SEQ ID NO: 115 - DNA, 27 bp, synthetic construct
  source 1..27: mol_type = other DNA
caggggtctggtgtctggtttgcttac

SEQ ID NO: 116 - DNA, 33 bp, synthetic construct; identical to SEQ ID NO: 100
  source 1..33: mol_type = other DNA

SEQ ID NO: 117 - skipped

SEQ ID NO: 118 - DNA, 27 bp, synthetic construct
  source 1..27: mol_type = other DNA
tttcaaggttcacatgttccgtacacg

SEQ ID NO: 119 - DNA, 75 bp, synthetic construct; identical to SEQ ID NO: 103
  source 1..75: mol_type = other DNA

SEQ ID NO: 120 - DNA, 51 bp, synthetic construct; identical to SEQ ID NO: 104
  source 1..51: mol_type = other DNA

SEQ ID NO: 121 - DNA, 120 bp, synthetic construct
  source 1..120: mol_type = other DNA
gagtttaattcagctctcaaatccagactgagcatcagcaaggacaactccaagagccaagttttcttaaaaatgaacagtctgcaaactgaagacacagccaggtactactgtgtcaga

SEQ ID NO: 122 - DNA, 24 bp, synthetic construct
  source 1..24: mol_type = other DNA
tggggccaagggactctggtcagt

SEQ ID NO: 123 - DNA, 78 bp, synthetic construct; identical to SEQ ID NO: 75
  source 1..78: mol_type = other DNA

SEQ ID NO: 124 - DNA, 51 bp, synthetic construct
  source 1..51: mol_type = other DNA
ttagaatggtacctgcagaaaccaggccagtctccaaagctcctgatttac

SEQ ID NO: 125 - DNA, 108 bp, synthetic construct
  source 1..108: mol_type = other DNA
aaccgattttctggggtcccagacaggttcagtggcagtggatcaggcacagatttcacactcaagatcagcagagtggaggctgaggatctgggagtttattactgc

SEQ ID NO: 126 - DNA, 30 bp, synthetic construct; identical to SEQ ID NO: 78
  source 1..30: mol_type = other DNA

SEQ ID NO: 127 - DNA, 342 bp, synthetic construct
  source 1..342: mol_type = other DNA
caggtgcagctgaaggagtcaggacctggcctggtggcgccctcacagagcctgtccatcacatgtaccgtctcagggttctcattaaacggctatggtgtaaactgggttcgccagcctccaggaaaggatctggagtggctgggaatgatctggggtgatggaatcacagagtttaattcagctctcaaatccagactgagcatcagcaaggacaactccaagagccaagttttcttaaaaatgaacagtctgcaaactgaagacacagccaggtactactgtgtcagacaggggtctggtgtctggtttgcttactggggccaagggactctggtcagt

SEQ ID NO: 128 - DNA, 336 bp, synthetic construct
  source 1..336: mol_type = other DNA
gatgttttggtgacccaaactccactctccctgcctgtcagtcttggagatcaagcctccatctcttgcagatctagtcagaccattgtacatagtaatggaaacacctatttagaatggtacctgcagaaaccaggccagtctccaaagctcctgatttacaaagtttccaaccgattttctggggtcccagacaggttcagtggcagtggatcaggcacagatttcacactcaagatcagcagagtggaggctgaggatctgggagtttattactgctttcaaggttcacatgttccgtacacgttcggaggggggaccaagctggaaataaaa

SEQ ID NO: 129 - protein, 181 aa, synthetic construct
MRGAARLGRPGRSCLPGARGLRAPPPPPLLLLLALLPLLPAPGAAAAPAPRPPELQSASAGPSVSLYLSEDEVRRLIGLDAELYYVRNDLISHYALSFSLLVPSETNFLHFTWHAKSKVEYKLGFQVDNVLAMDMPQVNISVQGEVPRTLSVFRVELSCTGKVDSEVMILMQLNLTVNSSK

SEQ ID NO: 130 - protein, 10 aa, synthetic construct
PAPRPPELQS

SEQ ID NO: 131 - protein, 214 aa, synthetic construct
MRAGRGGVPGSGGLRAPPPPLLLLLLAMLPAAAPRSPALAAAPAGPSVSLYLSEDEVRRLLGLDAELYYVRNDLISHYALSFNLLVPSETNFLHFTWHAKSKVEYKLGFQVDNFVAMGMPQVNISAQGEVPRTLSVFRVELSCTGKVDSEVMILMQLNLTVNSSKNFTVLNFKRRKMCYKKLEEVKTSALDKNTSRTIYDPVHAAPTTSTRVFY

SEQ ID NO: 132 - protein, 230 aa, synthetic construct
MRGAARLGRPGRSCLPGARGLRAPPPPPLLLLLALLPLLPAPGAAAAPAPRPPELQSASAGPSVSLYLSEDEVRRLIGLDAELYYVRNDLISHYALSFSLLVPSETNFLHFTWHAKSKVEYKLGFQVDNVLAMDMPQVNISVQGEVPRTLSVFRVELSCTGKVDSEVMILMQLNLTVNSSKNFTVLNFKRRKMCYKKLEEVKTSALDKNTSRTIYDPVHAAPTTSTRVFY

SEQ ID NO: 133 - protein, 97 aa, synthetic construct
QVQLKESGPGLVAPSQSLSITCTVSGFSLTGYGVNWVRQPPGKGLEWLGMIWGDGSTDYNSALKSRLSISKDNSKSQVFLKMNSLQTDDTARYYCAR

SEQ ID NO: 134 - protein, 100 aa, synthetic construct
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVP

SEQ ID NO: 135 - protein, 98 aa, synthetic construct
QVQLQQSGPELVKPGASVKLSCKASGYTFTSYDINWVKQRPGQGLEWIGWIYPRDGSTKYNEKFKGKATLTVDTSSSTAYMELHSLTSEDSAVYFCAR

SEQ ID NO: 136 - protein, 97 aa, synthetic construct
EIVLTQSPALMAASPGEKVTITCSVSSSISSSNLHWYQQKSETSPKPWIYGTSNLASGVPVRFSGSGSGTSYSLTISSMEAEDAATYYCQQWSSYPL